SEQUENCE LISTING (8 sequences)

SEQ ID NO: 1 - protein, 14 aa, Artificial Sequence
  Synthetic
CSVGTGGTNEKLFF

SEQ ID NO: 2 - DNA, 441 bp, Artificial Sequence
  Synthetic
gccttttctcaggggagaggccatcacttgaagatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgggacaggaggaactaatgaaaaactgttttttggcagtggaacccagctctctgtcttggaggacctgaacaaggtg

SEQ ID NO: 3 - protein, 147 aa, Artificial Sequence
  Synthetic
AFSQGRGHHLKMLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGTGGTNEKLFFGSGTQLSVLEDLNKV

SEQ ID NO: 4 - DNA, 45 bp, Artificial Sequence
  Synthetic
ctaatacgactcactatagggcaagcagtggtatcaacgcagagt

SEQ ID NO: 5 - DNA, 22 bp, Artificial Sequence
  Synthetic
ctaatacgactcactatagggc

SEQ ID NO: 6 - DNA, 23 bp, Artificial Sequence
  Synthetic
gcacaccagtgtggccttttggg

SEQ ID NO: 7 - DNA, 53 bp, Artificial Sequence
  Synthetic
cctatcccctgtgtgccttggcagtctcagaagcagtggtatcaacgcagagt

SEQ ID NO: 8 - DNA, 60 bp, Artificial Sequence
  Synthetic
ccatctcatccctgcgtgtctccgactcagnnnnnnnaacacagcgacctcgggtgggaa

(19) United States
(12) Patent Application Publication
(10) Pub. No.: US 2015/0167084 A1
(43) Pub. Date: Jun. 18, 2015

(54) Quantitative Assessment of Human T-Cell Repertoire Recovery After Allogeneic Hematopoietic Stem Cell Transplantation

(71) Applicant: SLOAN KETTERING INSTITUTE FOR CANCER RESEARCH, New York, NY (US)

(72) Inventors: Eric G. Pamer, Guilford, CT (US); Jeroen W. J. van Heijst, Amstelveen (NL); Miguel-Angel Perales, New York, NY (US); Marcel R. M. Van den Brink, New York, NY (US)

(21) Appl. No.: 14/412,715

(22) PCT Filed: Jul. 3, 2013
(86) PCT No.: PCT/US2013/049404

§ 371 Date: Jan. 5, 2015

Related U.S. Application Data
(60) Provisional application No. 61/667,783, filed on Jul. 3, 2012.

(51) Int. Cl. C12Q 1/68; C12Q 1/70

(52) U.S. Cl. CPC C12Q 1/6883; C12Q 1/70; C12Q 2600/112; C12Q 2600/106; C12Q 2600/136

(57) ABSTRACT

A method and an apparatus are provided for determining T-cell repertoire recovery after allo-HSCT or identifying patients at high risk of infection. Combination of 5′-RACE PCR with deep sequencing was used to quantify TCR diversity in 33 individuals using a single oligonucleotide pair. Analysis of duplicate blood samples revealed highly reproducible detection of expanded TCR clonotypes. After 6 months, recipients of cord blood grafts without anti-thymocyte globulin therapy approximated the TCR diversity of healthy subjects, whereas recipients of T-cell-depleted peripheral blood stem cell grafts had a 28-fold and 14-fold lower CD4+ and CD8+ T-cell diversity, respectively. After 12 months, these differences had leveled out for the CD4+, but not the CD8+ T-cell compartment.

### CROSS-REFERENCE TO RELATED APPLICATIONS

This application claims priority to U.S. Provisional Application Ser. No. 61/667,783 filed on Jul. 3, 2012 the entire contents of which are hereby incorporated by reference as if fully set forth herein, under 35 U.S.C. §119(e).

### STATEMENT OF GOVERNMENTAL INTEREST

This invention was made with Government support under Contract No. HL069929, CA107096 and A1080455 awarded by the National Institutes of Health (NIH) and under Contract No. W81XWH-09-1-0294 awarded by US Department of Defense. The Government has certain rights in the invention.

### BACKGROUND OF THE INVENTION

Allo-HSCT is a potentially curative treatment for a variety of hematologic diseases, including lymphoid and myeloid malignancies. Prior to transplantation, patients undergo conditioning with chemotherapy with or without irradiation, which results in severe immunodeficiency that particularly for the T-cell compartment can take months or years to restore^{1,2}. This prolonged T-cell deficiency predisposes patients to infection and cancer relapse³⁻⁶. Strategies that improve T-cell reconstitution and recovery of high TCR diversity could therefore greatly reduce transplant-associated morbidity and mortality.

Restoration of TCR diversity after allo-HSCT heavily depends on the thymic generation of new naive T cells⁸⁻¹⁰. Thymic function, however, diminishes markedly after the onset of puberty, and, in the allo-HSCT setting, is further impaired due to conditioning-associated damage and graft-versus-host disease (GVHD)^{11,12}. Thus, it is unclear how well TCR diversity can be restored, particularly in older patients using existing methods.

### SUMMARY OF THE INVENTION

In a first set of embodiments, a method for determining T-cell receptorβ clonotype diversity and frequency in a subject includes obtaining a blood sample from the subject and isolating CD4⁺ and CD8⁺ T-lymphocytes, or subsets of CD4⁺ and CD8⁺ T-lymphocytes. The method further comprises extracting total RNA from the cells isolated, generating cDNA from the total RNA, amplifying the cDNA, and sequencing the amplified cDNA. The method still further includes identifying T-cell receptor β clonotypes in the cDNA sequences and quantifying the diversity of the clonotypes and the clonotype frequency of each clonotype in the sample.

In a second set of embodiments, a method for determining a change in T-cell receptor β clonotype diversity and frequency in a subject over time includes obtaining a first blood sample from the subject at a first time point and obtaining a second blood sample at a second later time point. The method also includes isolating CD4⁺ and CD8⁺ T-lymphocytes or subsets of the CD4⁺ and CD8⁺ T-lymphocytes from each sample. The method further includes extracting total RNA from the cells isolated for each sample, generating cDNA from the total RNA for each sample, amplifying the cDNA for each sample, and sequencing the amplified cDNA for each sample. The method still further includes identifying T-cell receptor β clonotypes in the cDNA sequences and quantifying the diversity of the clonotypes and frequency of each clonotype in each sample. The method yet further includes determining, based on at least one of the diversity of the clonotypes in each sample or the frequency of at least one clonotype in each sample, whether there is a statistically significant increase in T-cell receptor β clonotype diversity or frequency at the second time point, or whether T-cell receptor β clonotype diversity or frequency is not statistically significantly changed at the second time point, or whether there is a statistically significant decrease in T-cell receptor β clonotype diversity or frequency at the second time point.

### BRIEF DESCRIPTION OF DRAWINGS

FIG. 1. Quantifying T-cell repertoire recovery after allo-HSCT. (a) Vβ gene usage of TCRs recovered from two separately processed blood samples of TCD patient #1 (TCD #1—A and B) as well as a representative healthy donor (Healthy #1—A and B). The 10 most frequent Vβ genes in TCD #1 are indicated in color, the remaining 38 Vβ genes are grouped in black. Nomenclature is according to the ImMunoGeneTics information system (IMGT). Number of reads: TCD #1, A (4,858) and B (11,044); Healthy #1, A (3,318) and B (5,009). (b) Digital CDR3 size spectratype plots of total TCRβ sequences from TCD #1 (15,902 reads) and Healthy #1 (8,327 reads). CDR3β length is defined as all amino acids (AA) in between the conserved 5′ cysteine and 3′ phenylalanine of the CDR3β region. (c) Clonotype distribution plots of total TCR sequences from TCD #1 and Healthy #1. Each diamond represents a distinct CDR3β AA sequence. (d) Dot plots comparing the clonotype distribution of two blood samples (A and B) from TCD #1 and Healthy #1. Each dot represents a distinct TCRβ clonotype. Dot opacity reflects multiple clonotypes of the same frequency. Values in the upper right corner depict the Pearson correlation. (e) TCR diversity of TCD #1, as well as the average TCR diversity of four individually-sequenced healthy subjects (Healthy #1-4). Error bars depict 95% confidence intervals.

FIG. 2. T-cell repertoire dynamics during the first year of allo-HSCT. (a) Vβ gene usage of TCRs recovered from TCD #1 at indicated time points after transplant. Number of reads: day 138 (15,902); day 147 (10,732); day 194 (11,220) and day 377 (3,980). (b) Dot plots comparing the clonotype distribution of two blood samples obtained on the same day from TCD #1 at the indicated time points. Number of reads: day 147, A (5,644) and B (5,088); day 194, A (4,445) and B (6,775); day 377, A (2,607) and B (1,373). (c) Dot plots comparing the clonotype distribution of blood samples obtained on different days from TCD #1 at the indicated time points. The red clonotype (Vβ 29.1/CDR3β CSVGTGGTNEKLFF) is specific for the HLA-A2-restricted BMLF1₂₈₀ epitope from EBV. The cyan clonotype was below the limit of detection on days 138 and 147, comprised 28% of the T-cell repertoire on day 194 and was again below the limit of detection on day 377. (d) Similarity of T-cell repertoires recovered from blood samples of TCD #1 obtained either on the same day (A vs. B) or on different days (D1 vs. D2). Values represent the Pearson correlation. Horizontal bars depict group mean. (e) Diversity of T-cell repertoires recovered from TCD #1 at the indicated time points. Error bars depict 95% confidence intervals.

FIG. 3. T-cell repertoire recovery by three different stem cell sources 6 and 12 months after allo-HSCT. Shown are representative clonotype distribution plots of CD4⁺ and CD8⁺ T cells obtained at either 6 or 12 months after conventional (Conv) or T-cell-depleted (TCD) peripheral blood stem cell transplantation, or double-unit umbilical cord blood (DUCB) transplantation. Healthy represents age-matched healthy subjects. (a) Clonotype distribution plots of Conv #2 (6 months; in red) and Conv #3 (12 months; in blue). Values in the lower-left corner depict the TCR diversity. Number of reads: Conv #2, CD4 (3,379) and CD8 (1,985); Conv #3, CD4 (1,954) and CD8 (1,515). (b) Clonotype distribution plots of TCD #6 (6 months; in red) and TCD #10 (12 months; in blue). Number of reads: TCD #6, CD4 (793) and CD8 (2,141); TCD #10, CD4 (2,889) and CD8 (694). (c) Clonotype distribution plots of DUCB #4 (6 months; in red) and DUCB #6 (12 months; in blue). Number of reads: DUCB #4, CD4 (2,312) and CD8 (1,138); DUCB #6, CD4 (2,173) and CD8 (680). (d) Clonotype distribution plots of Healthy #1. Number of reads: CD4 (2,856) and CD8 (800). (e) CD4⁺ T-cell diversity of indicated groups. Symbols depict individual subjects, bars depict group mean. *P=0.033. (f) CD8⁺ T-cell diversity of indicated groups. *P=0.012.

FIG. 4. T-cell repertoire recovery after allo-HSCT as a function of clinical variables. CD4⁺ and CD8⁺ T-cell diversity of 27 allo-HSCT recipients was divided according to clinical parameters that could influence T-cell repertoire recovery. (a) TCR diversity in patients that were either 21-48 years old (n=13) or 50-70 year old (n=14). (b) TCR diversity in patients that received either a matched related donor (MRD; n=6), a matched unrelated donor (MUD; n=6) or a mismatched unrelated donor (MMUD; n=15) transplantation. (c) TCR diversity in patients that either have not received (n=17) or have received (n=10) systemic steroid treatment. *P=0.023 and **P=0.006. (d) TCR diversity in patients that either had no or grade 1 acute GVHD (n=12) or grade 2 or grade 3 acute GVHD (n=15). ***P<0.001 and **P=0.003. (e) TCR diversity in patients that either have not been infected (n=15) or have been infected (n=12) with CMV or EBV. **P=0.004 and *P=0.033.

FIG. 5. Monitoring individual patients with poor T-cell repertoire recovery. Three patients identified after 12 months with very low CD4⁺ T-cell diversity (Conv #6/TCD #8) and very low CD8⁺ T-cell diversity (DUCB #7) were reanalyzed after 19-21 months. (a) Dot plots comparing the clonotype distribution of T cells isolated on different days from Conv #6 (CD4⁺ T cells; 218 days apart), TCD #8 (CD4⁺ T cells; 284 days apart) and DUCB #7 (CD8⁺ T cells; 225 days apart). Number of reads: Conv #6, day 377 (731) and day 594 (3,940); TCD #8, day 369 (610) and day 652 (688); DUCB #7, day 356 (3,932) and day 580 (3,825). (b) Dot plots comparing the clonotype distribution of CD8⁺ T cells isolated on different days from Healthy #1 (109 days apart), Healthy #3 (109 days apart) and Healthy #4 (299 days apart). Number of reads: Healthy #1, day 0 (800) and day 109 (1,120); Healthy #3, day 0 (2,267) and day 109 (1,508); Healthy #4, day 0 (917) and day 299 (1,068). (c) Clonotype distribution plots of CD4⁺ T cells isolated from Conv #6 (days 377 and 594) and TCD #8 (days 369 and 652) as well as and CD8⁺ T cells isolated from DUCB #7 (days 356 and 580). Values in the lower-left corner depict the TCR diversity.

FIG. 6. Repertoire analysis of unseparated T cells isolated from four healthy donors. (a) Vβ gene usage of total TCRβ sequences from Healthy nos. 1-4. All 48 Vβ genes were found, ranging from 17.8±6% for TRBV5-1 to 0.003±0.006% for TRBV16. Nomenclature is according to IMGT. TRBV4-2 or 4-3, 6-2 or 6-3 and 12-3 or 12-4 represent TCRs for which insufficient sequence information was available to assign the correct Vβ gene. Number of reads: 26,785. (b) Vβ gene usage of two separately processed blood samples (A and B) of Healthy nos. 1-4. The 10 most frequent Vβ genes found in TCD no. 1 at 138 days after transplant are indicated in color, the remaining 38 Vβ genes are grouped in black. Number of reads: Healthy no. 1, A (3,318) and B (5,009); Healthy no. 2, A (3,892) and B (4,045); Healthy no. 3, A (2,481) and B (1,494); Healthy no. 4, A (3,617) and B (2,929). (c) Digital CDR3 size spectratype plots of total TCRβ sequences from Healthy nos. 1-4. (d) Clonotype distribution plots of total TCRβ sequences from Healthy nos. 1-4. Each diamond represents a distinct CDR3β amino acid (AA) sequence. (e) Dot plots comparing the clonotype distribution of two blood samples (A and B) from Healthy nos. 1-4. Each dot represents a distinct TCRβ clonotype. Dot opacity reflects multiple clonotypes of the same frequency. Values in the upper right corner depict the Pearson correlation. (f) TCR diversity of Healthy nos. 1-4. Error bars depict 95% confidence intervals.

FIG. 7. TCR diversity of separated naïve and memory CD8+ T cells. Isolated mononuclear cells from Healthy no. 1 were sorted by flow cytometry into naïve (CD45RA+CD45RO—) and memory (CD45RA-CD45RO+) CD8+ T cells. Subsequently, TCR diversity of both fractions was determined. (a) Dot plot comparing the clonotype distribution of sorted naïve and memory CD8+ T cells. Virtual absence of overlapping clonotypes indicates high purity of the sort. (b) Clonotype distribution plots of sorted naïve and memory CD8+ T cells. (c) TCR diversity of sorted naïve and memory CD8+ T cells. Error bars depict 95% confidence intervals.

FIG. 8. Highly volatile T-cell repertoire of TCD no. 1 partly coincided with EBV reactivation. (a) Dot plots comparing the clonotype distribution of two blood samples obtained from TCD no. 1 at indicated days after transplant. The red clonotype (TRBV29-1 and CDR3β CSVGTGGTNEKLFF) is specific for the HLA-A2-restricted BMLF1280 epitope from EBV. This BMLF1-specific clonotype was below the limit of detection on day 138, comprised 2.9% of the repertoire on day 147 (making it the 9th most abundant clonotype at this timepoint), was again below the limit of detection on day 194, and reappeared at 0.1% of the repertoire on day 377. Number of reads: day 138 (15,902); day 147 (10,732); day 194 (11,220) and day 377 (3,980). (b) EBV reactivation of TCD no. 1 determined by PCR assay. Black dots depict timepoints of EBV PCR analysis. Dotted lines depict timepoints of T-cell repertoire analysis. Following detection of EBV reactivation on day 145, the patient was treated with rituximab in between days 151 and 173, which dropped EBV levels below the limit of detection (LOD; 500 copies ml-1) by day 158. Therefore, the T-cell repertoire analysis of day 147 corresponded to the peak of EBV infection detected in this patient. (c) Digital CDR3 size spectratype plot of all TCRs that used TRBV29-1 on day 147. Of all TCRs with a CDR3β length of 12 AA, the BMLF1-specific clonotype contributed 91.7%. Number of reads: 526.

FIG. 9. Differential recovery of CD4+ and CD8+ T-cell repertoires after allo-HSCT. To allow distinction between CD4+ and CD8+ T-cell repertoire recovery, CD4+ and CD8+ T cells were separated from peripheral blood of TCD no. 1 at day 377 after transplant. (a) Flow cytometry plots depicting the purity of CD4+ and CD8+ T cells after magnetic separation. Plots were gated on live, singlet, CD14− cells. Numbers depict percentage of gated cells. (b) Vβ gene usage of separated CD4+ and CD8+ T cells. The 10 most frequent Vβ genes are indicated in color, the remaining 38 Vβ genes are grouped in black. Of all 48 Vβ genes, 44 were found in the CD4+ T-cell compartment, whereas only 28 were found in the CD8+ T-cell compartment. Number of reads: CD4 (8,024) and CD8 (4,194). (c) Clonotype distribution plots of separated CD4+ and CD8+ T cells. While the CD4+ T-cell repertoire was relatively evenly distributed, with the most abundant clonotype comprising 5.1% of reads, the CD8+ T-cell repertoire was heavily skewed and contained four clonotypes that together comprised 85% of reads, of which the most abundant clonotype contributed 32%. (d) Dot plot comparing the clonotype distribution of separated CD4+ and CD8+ T cells. (e) TCR diversity of separated CD4+ and CD8+ T cells. Error bars depict 95% confidence intervals.

FIG. 10. Higher CD4+ T-cell diversity in cord blood recipients correlates with increased numbers of naïve CD4+ T cells. (a) Absolute number of naïve (CD45RA+) CD4+ T cells either 6 months (closed symbols) or 12 months (open symbols) after T-cell-depleted peripheral blood stem cell transplantation (TCD; in red) or double-unit umbilical cord blood transplantation (DUCB; in blue). *P=0.023. (b) Comparison of the number of naïve CD4+ T cells against TCR diversity for each patient. The positive Pearson correlation between both variables (r: 0.58) is statistically significant (P=0.007).

FIG. 11. Identification of four patients with normal T-cell counts, but very low TCR diversity at 12 months after allo-HSCT. (a) Comparison of absolute T-cell counts against TCR diversity either 6 months (triangles) or 12 months (circles) after conventional (Conv) or T-cell-depleted (TCD) peripheral blood stem cell transplantation, or double-unit umbilical cord blood (DUCB) transplantation. Top panel shows CD4+ T cells, bottom panel shows CD8+ T cells. Each symbol depicts an individual patient. Red circles highlight patients with normal T-cell counts, but particularly low TCR diversity compared to their group mean. (b) Clonotype distribution plots of Conv no. 5. Values in the lower-left corner depict the TCR diversity. This patient had a ˜80-fold lower CD4+ T-cell diversity compared to its group mean (1/Ds: 3,298). (c) Clonotype distribution plots of Conv no. 6. This patient had a ˜150-fold lower CD4+ T-cell diversity compared to its group mean. (d) Clonotype distribution plots of TCD no. 8. This patient had a ˜60-fold lower CD4+ T-cell diversity compared to its group mean (1/Ds: 1,871). (e) Clonotype distribution plots of DUCB no. 2. This patient had a ˜25-fold lower CD8+ T-cell diversity compared to its group mean (1/Ds: 159). Number of reads: Conv no. 5, CD4 (8,780) and CD8 (5,340); Conv no. 6, CD4 (731) and CD8 (8,920); TCD no. 8, CD4 (610) and CD8 (1,324); DUCB no. 7, CD4 (1,253) and CD8 (3,932).

FIG. 12. Relative stability of the T-cell repertoire in healthy donors. To assess CD4+ and CD8+ T-cell repertoire stability in healthy individuals, three healthy donors were reanalyzed either 109 days (Healthy nos. 1 and 3) or 299 days (Healthy no. 4) after the first timepoint. (a) Dot plots comparing the clonotype distribution of CD4+ and CD8+ T cells isolated on different days from Healthy nos. 1, 3 and 4. Repertoire overlap of the CD4+ T-cell compartment is very low because most clonotypes were not abundant enough to pass the threshold for physical presence in a second blood sample (˜0.16% of total). In contrast, repertoire overlap of the CD8+ T-cell compartment is substantial, although several abundant clonotypes were detected at the second timepoint that were not seen before (red clonotype in Healthy no. 1: 10.4% of reads on day 109; cyan clonotype in Healthy no. 4; 11.6% of reads on day 299). Number of reads: Healthy no. 1, CD4 (day 0: 2,856-day 109: 2,973) and CD8 (day 0: 800-day 109: 1,120); Healthy no. 3, CD4 (day 0: 3,345-day 109: 1,487) and CD8 (day 0: 2,267-day 109: 1,508); Healthy no. 4, CD4 (day 0: 5,846-day 299: 1,764) and CD8 (day 0: 917-day 299: 1,068). (b) Clonotype distribution plots of CD4+ and CD8+ T cells isolated on indicated days from Healthy nos. 1, 3 and 4. On average, TCR diversity of both T-cell compartments was highly stable and fluctuated within a ˜1.5-fold range.

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication. Second Edition (W.H. Freeman. N.Y., 1992); Lehninger, Biochemistry. Second Edition (Worth Publishers. New York, 1975): Strachan and Read. Human Molecular Genetics. Second Edition (Wiley-Liss, New York, 1999); Abbas et al, Cellular and Molecular Immunology, 6.sup.th edition (Saunders, 2007).

As used herein, “nucleic acid” means DNA, RNA and derivatives thereof. In some embodiments, the nucleic acid is single stranded. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, phosphodiester group modifications (e.g., phosphorothioates, phosphorodithioates, boranophosphonates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping moieties. A 2′ deoxy nucleic acid linker is a divalent nucleic acid compound of any appropriate length and/or internucleotide linkage wherein the nucleotides are 2′ deoxy nucleotides.

The terms “DNA” and “RNA” refer to deoxyribonucleic acid and ribonucleic acid, respectively.

Where a method disclosed herein refers to “amplifying” a nucleic acid, the term “amplifying” refers to a process in which the nucleic acid is exposed to at least one round of extension, replication, or transcription in order to increase (e.g., exponentially increase) the number of copies (including complimentary copies) of the nucleic acid. The process can be iterative including multiple rounds of extension, replication, or transcription. Various nucleic acid amplification techniques are known in the art, such as PCR amplification or rolling circle amplification.

A “primer” as used herein refers to a nucleic acid that is capable of hybridizing to a complimentary nucleic acid sequence in order to facilitate enzymatic extension, replication or transcription.

“Complementary,” as used herein, refers to the capacity for precise pairing of two nucleobases (e.g., A to T (or U), and G to C) regardless of where in the nucleic acid the two are located. For example, if a nucleobase at a certain position of nucleic acid is capable of hydrogen bonding with a nucleobase at a certain position of another nucleic acid, then the position of hydrogen bonding between the two nucleic acids is considered to be a complementary position. Nucleic acids are “substantially complementary” to each other when a sufficient number of complementary positions in each molecule are occupied by nucleobases that can hydrogen bond with each other. Thus, the term “substantially complementary” is used to indicate a sufficient degree of precise pairing over a sufficient number of nucleobases such that stable and specific binding occurs between the nucleic acids. The phrase “substantially complementary” thus means that there may be one or more mismatches between the nucleic acids when they are aligned, provided that stable and specific binding occurs. The term “mismatch” refers to a site at which a nucleobase in one nucleic acid and a nucleobase in another nucleic acid with which it is aligned are not complementary. The nucleic acids are “perfectly complementary” to each other when they are fully complementary across their entire length.

The phrase “amino acid” as used herein refers to any of the twenty naturally occurring amino acids as well as any modified amino acids. Modifications can include natural processes such as posttranslational processing, or chemical modifications which are known in the art. Modifications include, but are not limited to, phosphorylation, ubiquitination, acetylation, amidation, glycosylation, covalent attachment of flavin, ADP-ribosylation, cross linking, iodination, methylation, and the like.

The words “protein”, “peptide”, and “polypeptide” are used interchangeably to denote an amino acid polymer or a set of two or more interacting or bound amino acid polymers. Rapid Amplification of cDNA Ends (RACE) is a technique used in molecular biology to obtain the full length sequence of an RNA transcript found within a cell. RACE results in the production of a cDNA copy of the RNA sequence of interest, produced through reverse transcription, followed by PCR amplification of the cDNA copies (see RT-PCR). The amplified cDNA copies are then sequenced and, if long enough, should map to a unique mRNA already described, the full sequence of which is known. RACE can provide the sequence of an RNA transcript from a small known sequence within the transcript to the 5′ end (5′ RACE-PCR). The first step in RACE is to use reverse transcription to produce a cDNA copy of a region of the RNA transcript. In this process, an unknown end portion of a transcript is copied using a known sequence from the center of the transcript. The copied region is bounded by the known sequence, and either the 5′ or 3′ end. 5′ RACE-PCR begins using mRNA as a template for a first round of cDNA synthesis (or reverse transcription) reaction using an anti-sense (reverse) oligonucleotide primer that recognizes a known sequence in the gene of interest; the primer is called a gene specific primer (GSP), and it copies the mRNA template in the 3′ to the 5′ direction to generate a specific single-stranded cDNA product. Following cDNA synthesis, the enzyme terminal deoxynucleotidyl transferase (TdT) is used to add a string of identical nucleotides, known as a homopolymeric tail, to the 3′ end of the cDNA. (There are some other ways to add the 3′-terminal sequence for the first strand of the de novo cDNA synthesis which are much more efficient than homopolymeric tailing, but the sense of the method remains the same). A PCR reaction is then carried out, which uses a second anti-sense gene specific primer (GSP2) that binds to the known sequence, and a sense (forward) universal primer (UP) that binds the homopolymeric tail added to the 3′ ends of the cDNAs to amplify a cDNA product from the 5′ end.

“Deep sequencing” is used herein in conformity with the ordinary meaning of the term in the art, i.e., high-throughput sequencing methodology such as the massively parallel sequencing methodologies for example using Illumina and Roche/454. Deep sequencing can analyze tens of millions of reads in parallel.

“Clonality” as used herein means a measure of the degree to which the distribution of clonotype abundances among clonotypes of a repertoire is skewed to a single or a few clonotypes. Roughly, clonality is an inverse measure of clonotype diversity.

“Clonotype” means a recombined nucleotide sequence of a T cell encoding a T cell receptor (TCR), or a portion thereof. In one aspect, a collection of all the distinct clonotypes of a population of lymphocytes of an individual is a repertoire of such population, e.g. Arstila et al. Science, 286: 958-961 (1999); Yassai et al. Immunogenetics, 61: 493-502 (2009); Kedzierska et al, Mol. Immunol., 45(3): 607-618 (2008); and the like. In one aspect, clonotypes of a repertoire comprises any segment of nucleic acid common to a T cell population which has undergone somatic recombination during the development of TCRs, including normal or aberrant (e.g. associated with cancers) precursor molecules thereof, including, but not limited to any of the following: an immunoglobulin heavy chain (IgH) or subsets thereof (e.g. an IgH variable region, CDR3 region, or the like), incomplete IgH molecules, an immunoglobulin light chain or subsets thereof (e.g. a variable region, CDR region, or the like). T cell receptor .alpha. chain or subsets thereof, T cell receptor .beta. chain or subsets thereof (e.g. variable region, CDR3, V(D)J region, or the like), a CDR (including CDR1, CDR2 or CDR3, of either TCRs or BCRs, or combinations of such CDRs). V(D)J regions of either TCRs or BCRs, hypermutated regions of IgH variable regions, or the like.

As used herein, “clonotype profile.” or “repertoire profile,” is a tabulation of clonotypes of a sample of T cells (such as a peripheral blood sample containing such cells) that includes substantially all of the repertoire's clonotypes and their relative abundances. “Clonotype profile,” “repertoire profile,” and “repertoire” are used herein interchangeably. The term “repertoire,” means a repertoire measured from a sample of T lymphocytes). In one aspect of the invention, clonotypes comprise portions of a TCRβ. chain. In other aspects of the invention, clonotypes may be based on other recombined molecules, such as immunoglobulin light chains or TCR.alpha chains, or portions thereof.

“Repertoire”, or “immune repertoire”, means a set of distinct recombined nucleotide sequences that encode T cell receptors (TCRs), or fragments thereof, in a population of T-lymphocytes of an individual, wherein the nucleotide sequences of the set have a one-to-one correspondence with distinct lymphocytes or their clonal subpopulations for substantially all of the lymphocytes of the population. In one aspect, a population of lymphocytes from which a repertoire is determined is taken front one or more tissue samples, such as one or more blood samples.

“Immunosuppression” can occur in, for example, malnutrition, aging, many types of cancer (such as leukemia, lymphoma, multiple myeloma), sepsis and certain chronic infections such as acquired immunodeficiency syndrome (HIV/AIDS). The unwanted effect in immunosuppression is immunodeficiency that results in increased susceptibility to pathogens such as bacteria and virus. In the context of the present invention “immunodeficiency” or “immune compromised/immunocompromised” are used interchangeably and refer to T-cell deficiencies that cause the disorders. These include marrow and other transplants, AIDS, HIV, Cancer chemotherapy, lymphoma and subjects undergoing glucocorticoid therapy, infections caused by intracellular pathogens including Herpes simplex virus, Mycobacterium, Listeria, and intracellular fungal infections. A person who has an immunodeficiency of any kind is said to be immunocompromised. An immunocompromised person may be particularly vulnerable to opportunistic infections, in addition to normal infections that could affect everyone.

Autoimmune diseases include but are not limited to the following: Acute Disseminated Encephalomyelitis (ADEM); Acute necrotizing hemorrhagic leukoencephalitis; Addison's disease; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune hepatitis; Autoimmune hyperlipidemia; Autoimmune immunodeficiency; Autoimmune inner ear disease (AIED); Autoimmune myocarditis; Autoimmune oophoritis; Autoimmune pancreatitis; Autoimmune retinopathy; Autoimmune thrombocytopenic purpura (ATP); Autoimmune thyroid disease; Autoimmune urticarial; Axonal & neuronal neuropathies; Balo disease; Behcet's disease; Bullous pemphigoid; Cardiomyopathy; Castleman disease; Celiac disease; Chagas disease; Chronic fatigue syndrome**; Chronic inflammatory demyelinating polyneuropathy (CIDP); Chronic recurrent multifocal ostomyelitis (CRMO); Churg-Strauss syndrome; Cicatricial pemphigoid/benign mucosal pemphigoid; Crohn's disease; Cogans syndrome; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic esophagitis; Eosinophilic fasciitis; Erythema nodosum; Experimental allergic encephalomyelitis; Evans syndrome; Fibromyalgia**; Fibrosing alveolitis; Giant cell arteritis (temporal arteritis); Giant cell myocarditis; Glomerulonephritis; Goodpasture's syndrome; Granulomatosis with Polyangiitis (GPA) (formerly called Wegener's Granulomatosis); Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropathy; IgG4-related sclerosing disease; Immunoregulatory lipoproteins; Inclusion body myositis; Interstitial cystitis; Juvenile arthritis; Juvenile diabetes (Type 1 diabetes); Juvenile myositis; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatic; Polymyositis; Postmyocardial infarction syndrome; Postpericardiotomy syndrome; Progesterone dermatitis; Primary biliary cirrhosis; Primary sclerosing cholangitis; Psoriasis; Psoriatic arthritis; Idiopathic pulmonary fibrosis; Pyoderma gangrenosum; Pure red cell aplasia; Raynauds phenomenon; Reactive Arthritis; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sjogren's syndrome; Sperm & testicular autoimmunity; Stiff person syndrome; Subacute bacterial endocarditis (SBE); Susac's syndrome; Sympathetic ophthalmia; Takayasu's arteritis; Temporal arteritis/Giant cell arteritis; Thrombocytopenic purpura (TTP); Tolosa-Hunt syndrome; Transverse myelitis; Type 1 diabetes; Ulcerative colitis; Undifferentiated connective tissue disease (UCTD); Uveitis; Vasculitis; Vesiculobullous dermatosis; Vitiligo; and Wegener's granulomatosis (now termed Granulomatosis with Polyangiitis (GPA).

### DETAILED DESCRIPTION

Delayed T-cell recovery and restricted T-cell receptor (TCR) diversity after allogeneic hematopoietic stem cell transplantation (allo-HSCT) are related to the increased risks of infection and cancer relapse. Measuring T-cell receptor diversity is challenging and generally requires numerous molecular assays that must be performed in parallel in order to obtain a rough estimate of the receptor repertoire. A simple and rapid method that enables T-cell receptor repertoire determination is needed.

New methods for determining T-cell receptor β clonotype diversity and frequency have been discovered that permit 1) a comparison of the clonotype diversity between a subject having a disease associated with immunosuppression or immunodeficiency (such as in a subject that has received an allo-HSCT) or an autoimmune disease and a healthy subject, 2) monitoring recovery of T-cell receptor β clonotype diversity in an immunosuppressed subject such as a cancer patient, to identify, inter alia, subjects at risk of cancer relapse, 3) determining if a therapeutic regimen is causing an increase or decrease or no change in clonogype diversity and frequency over the course of therapy as a way to determine treatment efficacy, and 4) screening test agents to identify a test agent that increases T-cell receptor β clonotype diversity. A summary of these and other new methods is set forth in the Summary of the Invention.

### OVERVIEW

Over the past two decades, several strategies have been developed to probe human TCR diversity. One strategy aims to identify the presence of different TCR families, by using flow cytometry or PCR to determine the usage of different TCR variable (V) genes^{13,14}. A second strategy, called CDR3 size spectratyping, aims to determine polyclonality of the repertoire, by using fluorescent primers to measure length variation of the CDR3 region within each TCR V family^{15,16}. Spectratyping in particular has been useful to document substantial abnormalities in T-cell repertoire composition after allo-HSCT¹⁷⁻¹⁹. However, as neither of these strategies is able to measure the frequency of individual TCRs, they can only provide an estimate of repertoire complexity. With the advent of deep sequencing technology, it has now become possible to directly measure TCR diversity with high resolution²⁰⁻²⁶. Embodiments of the present invention incorporate deep sequencing to address two fundamental questions related to T-cell reconstitution after allo-HSCT: how TCR diversity recovers I) over time and II) as a function of different stem cell sources (i.e. different types of transplants)^{27,28}.

Embodiments of the invention are directed to a method to reproducibly and accurately measure human TCR diversity. In an embodiment 5′-RACE PCR is combined with deep sequencing, to assess the entire TCR receptor β repertoire using a single oligonucleotide pair, thereby eliminating amplification bias. This contrasts with TCR sequencing methods based on gDNA²⁰⁻²³, which have to use many different oligonucleotides for amplification, making some degree of bias unavoidable. While 5′-RACE PCR provides a clear advantage, a limitation is that it requires RNA, and thus changes in TCR transcription could skew the frequency of particular clonotypes. Although Roche/454 sequencing was used in the Examples herein, the new methods can be readily adaptable to other platforms with greater sequence coverage, which could help to identify infrequent TCRs. The Illumina MiSeq platform provides deeper sequencing capacity, with the ability to determine T-cell receptor diversity and the presence of T cell clonotypes in individuals with a broad repertoire.

Experiments were conducted to determine T-cell repertoire recovery in allo-HSCT recipients over time, in whom limited TCR diversity is linked to susceptibility to infection and cancer relapse. Although significant improvement in TCR diversity in allo-HSCT subjects over time, there was substantial variability in the rate of recovery between different stem cell sources, including DUCB, TCD and unmanipulated peripheral blood stem cell transplants. Most notably, DUCB recipients had a 28-fold higher CD4⁺ T-cell diversity compared to TCD recipients after 6 months. It is important to note that this is consistent with clinical findings, which have shown that after 6 months, DUCB recipients have a low incidence of infection,³³ higher CD4⁺ T-cell numbers and a lower rate of leukemia relapse²⁷ than TCD recipients.^{33,34} Although many variables could contribute to this differential repertoire recovery, at least partially it can be explained by the fact that DUCB recipients receive ˜7,000-fold more T cells in their graft and transplantation is performed without T-cell-depleting regimens.³³

Besides allo-HSCT, this method should be useful to characterize T-cell immunity in other clinical settings of immune deficiency, autoimmunity and tumor immunity. Ultimately, the ability to measure T-cell repertoire complexity with great precision should guide the way for novel therapeutic approaches aimed at immune regeneration. Hence certain embodiments are directed to screening test agents to identify those that increase T cell diversity in cultured T cells (CD4 and CD8) taken from an immunosuppressed subject.

### SUMMARY OF RESULTS

T cells typically express only one TCRβ chain, making sequence analysis of TCRβ cDNA a useful measure of TCR diversity. However, in some embodiments T cell diversity is measured by sequence analysis of TCRalpha or Igg.

Analysis of the TCRβ repertoire following T-cell-depleted (TCD) peripheral blood stem cell transplantation (TCD) was highly reproducible using 5′-RACE PCR, which amplified all 48 Vβ genes using a single oligonucleotide pair. See Table 1 which shows that the entire TCRβ repertoire was covered.

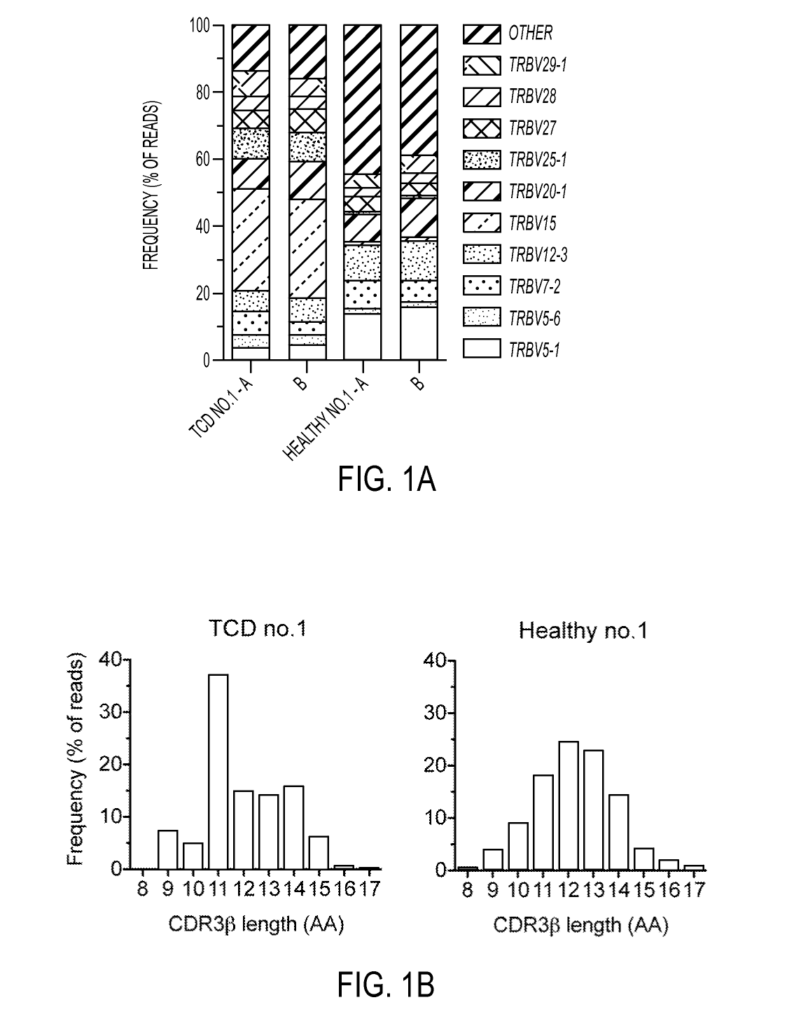

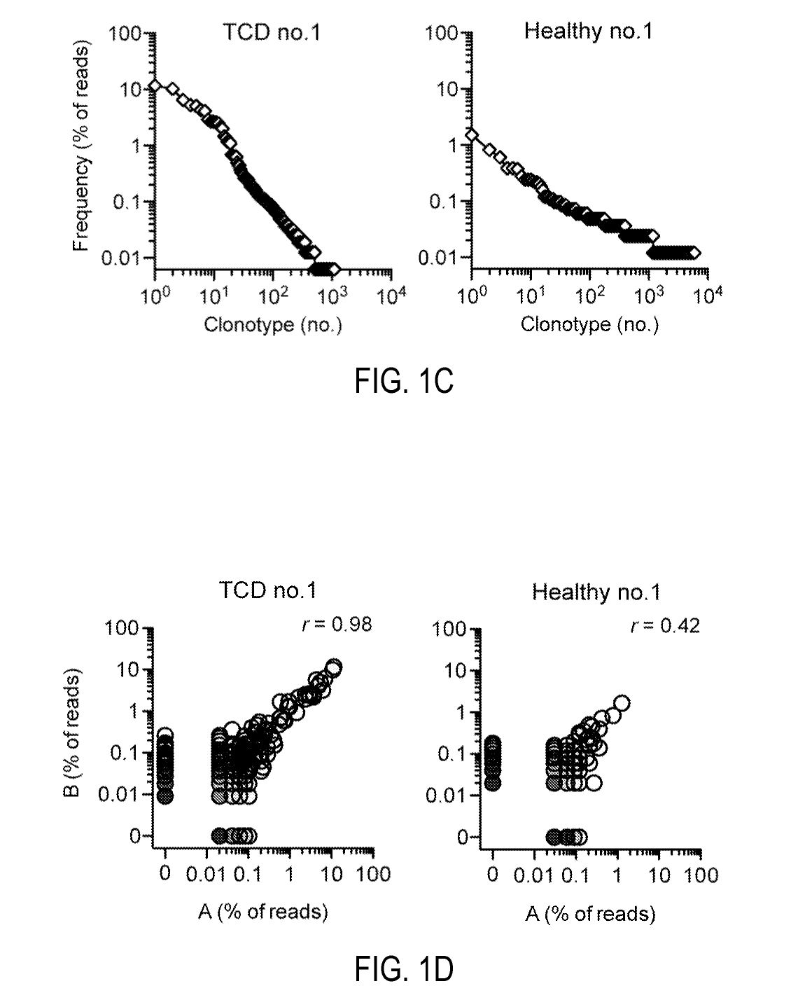

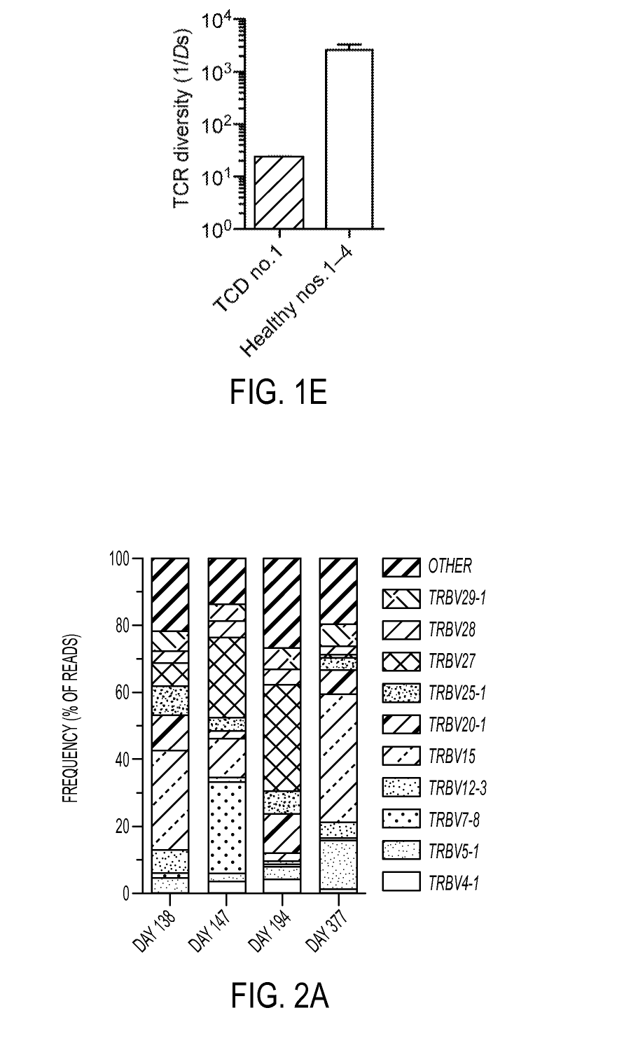

Comparison of two blood samples from a single TCB transplant patient (TCB #1) showed a highly reproducible pattern of Vβ usage, which differed markedly from healthy subjects (FIG. 1a) showing that there were substantial clonal expansions in the patient's repertoire, as was confirmed by digital CDR3 size spectratype profiles (FIG. 1b).

Analysis of the TCB #1 patient's repertoire at 138 days after transplant revealed a very low TCR diversity (1/Ds: 23), which was more than 100-fold lower than the average diversity of four healthy subjects (1/Ds: 2,525; FIG. 1e) indicating that the T-cell repertoire was highly restricted.

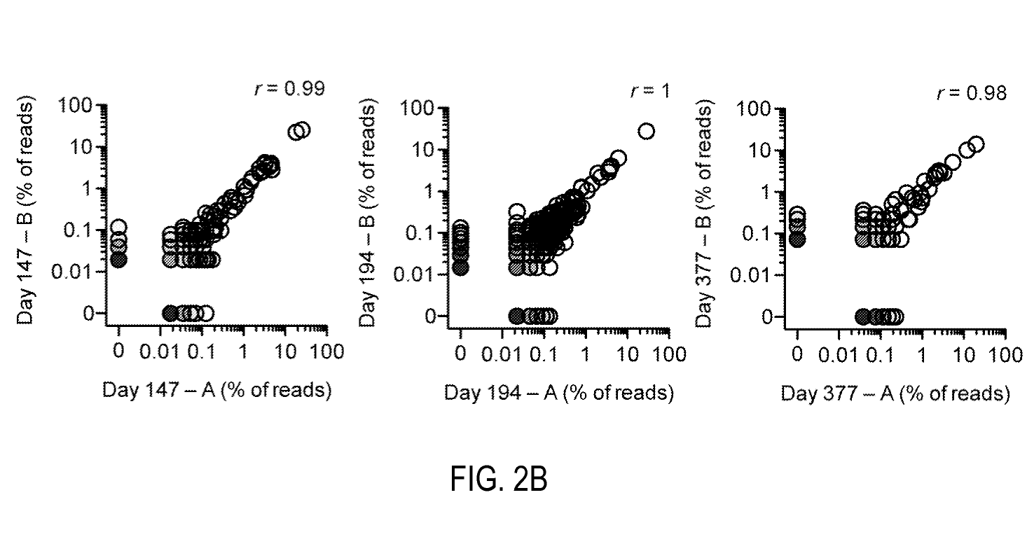

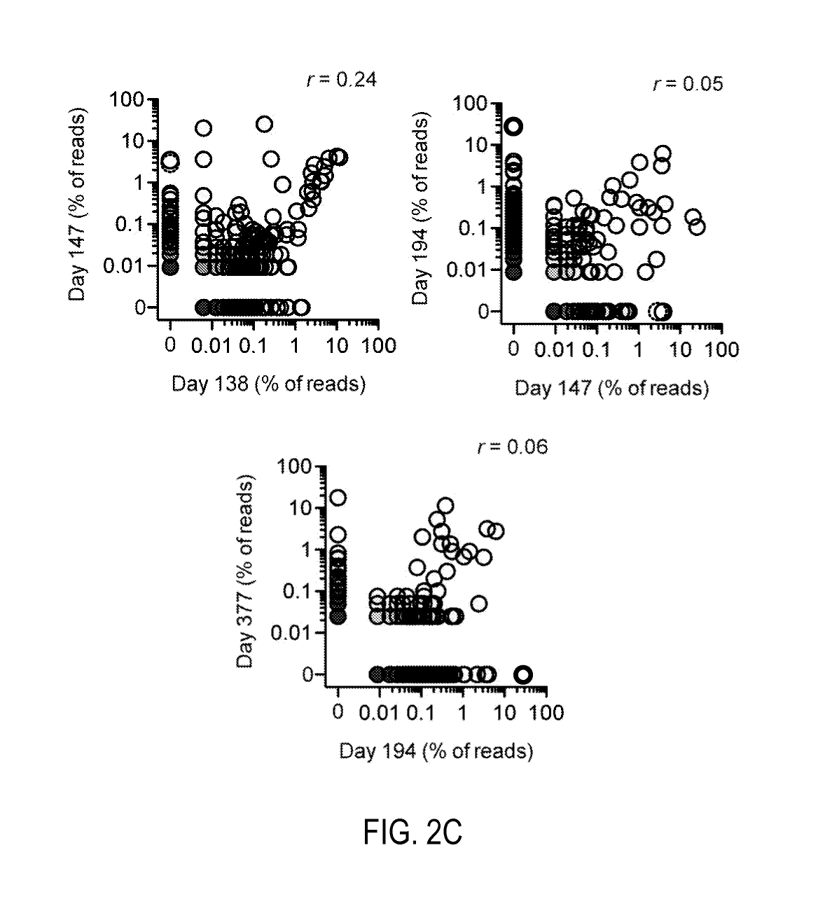

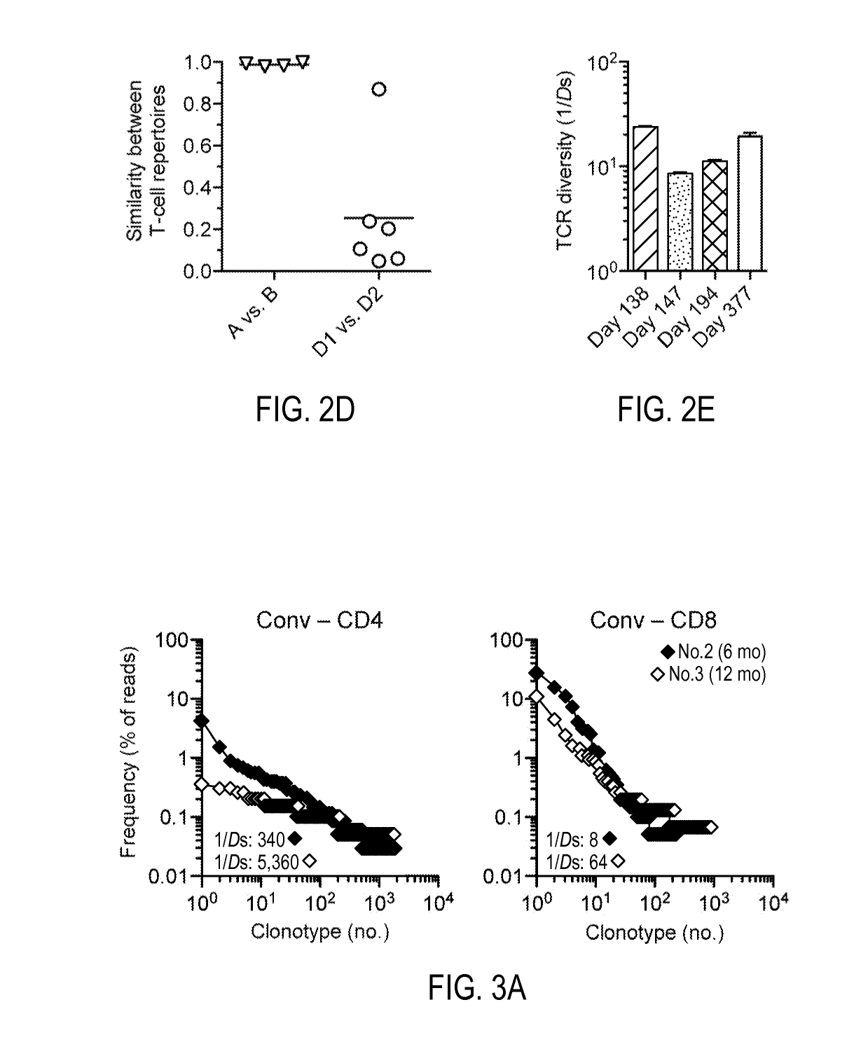

Despite profound changes in repertoire composition, TCR diversity did not increase over time (1/Ds: 23 and 19 for days 138 and 377, respectively; FIG. 2e) in the TCB #1 patient. Therefore, between 4.5 and 12.5 months after transplant, the complexity of this patient's T-cell repertoire did not improve.

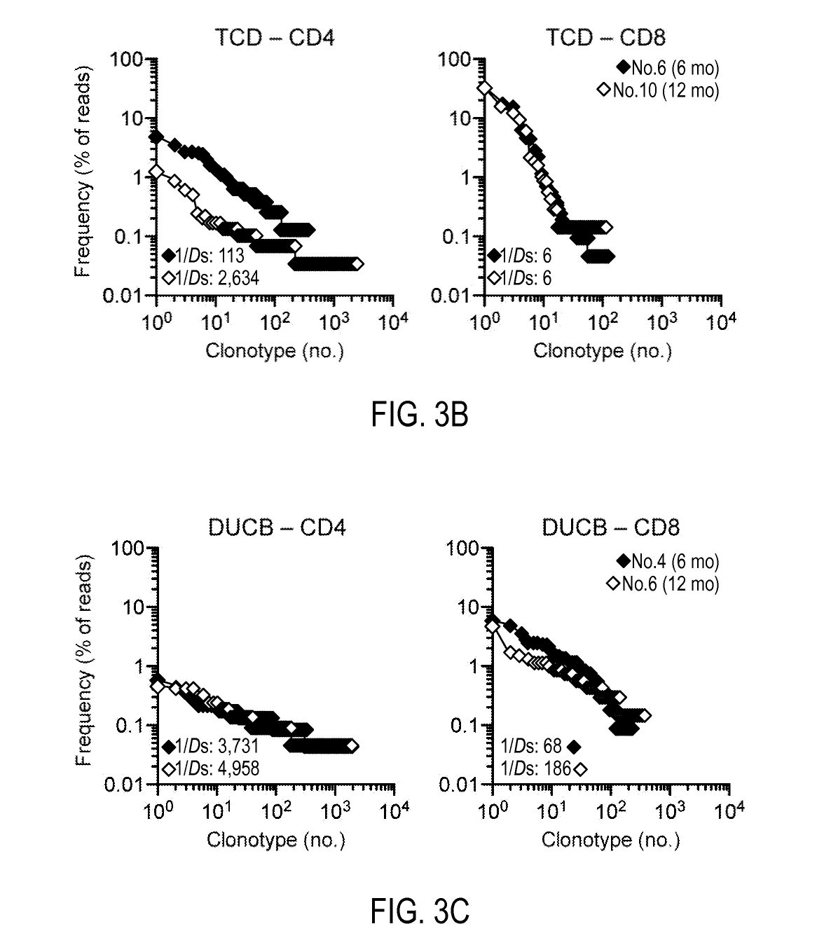

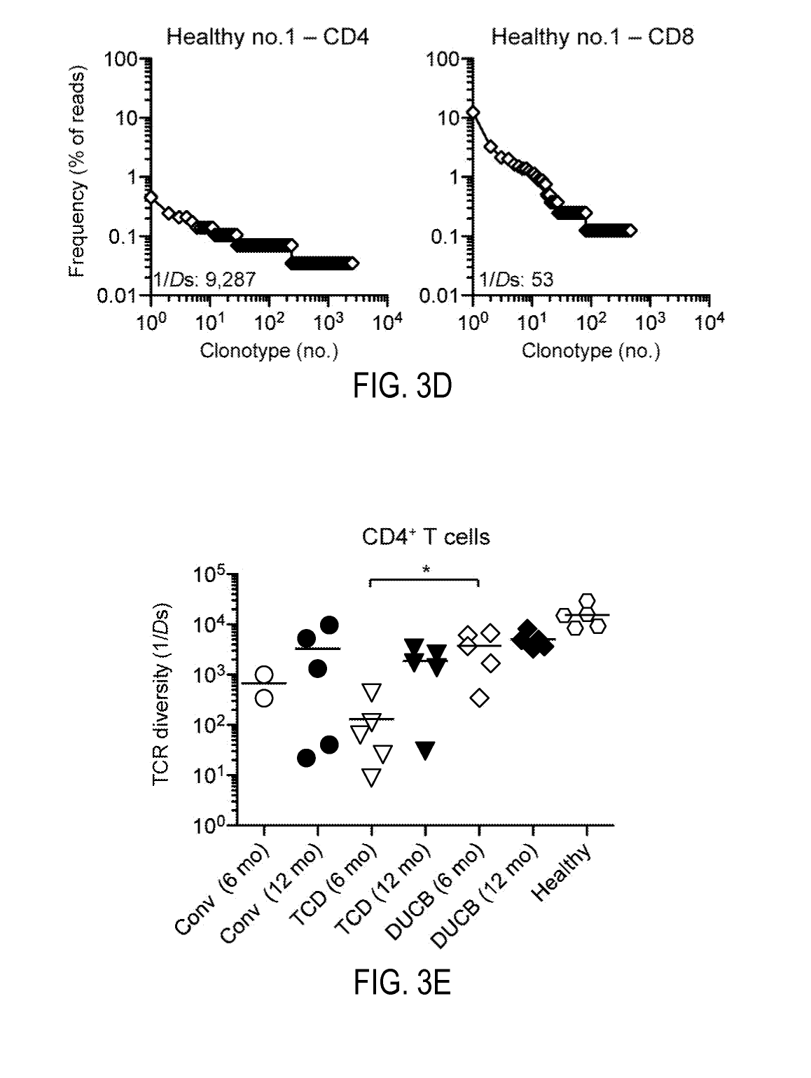

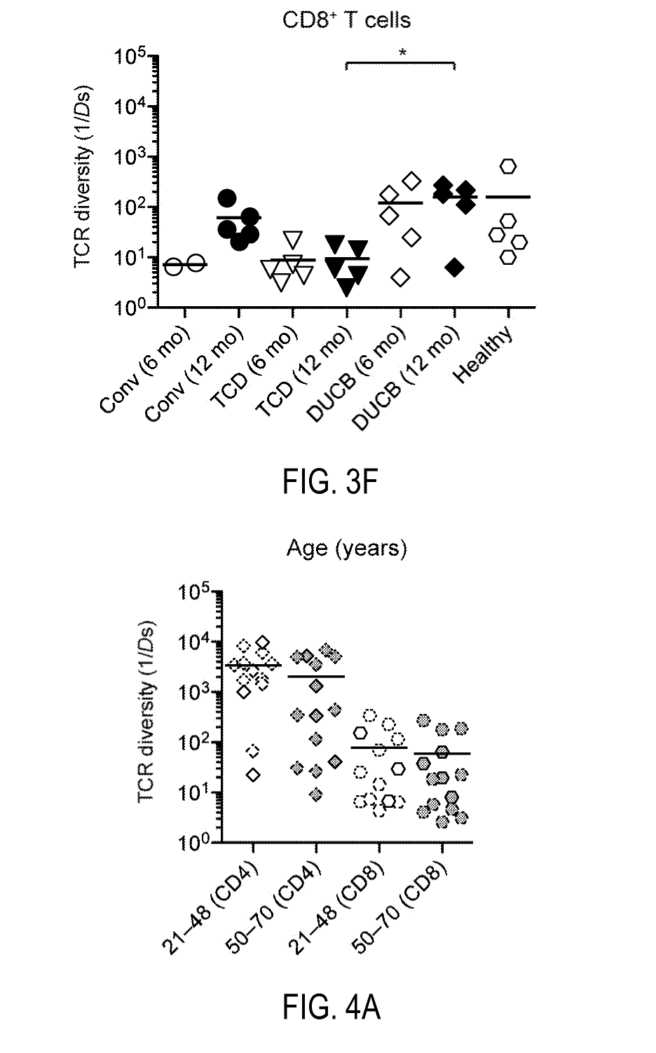

TCR diversity in 27 patients at either 6 or 12 months after conventional (Conv) or TCD peripheral blood stem cell transplantation, or double-unit umbilical cord blood (DUCB) transplantation (FIG. 3a-c) showed:

I) For all stem cell sources as well as for healthy subjects, CD4⁺ T-cell diversity was ˜50-times higher than CD8⁺ T-cell diversity (1/Ds: 4,665 and 81, respectively; FIG. 3e,f).

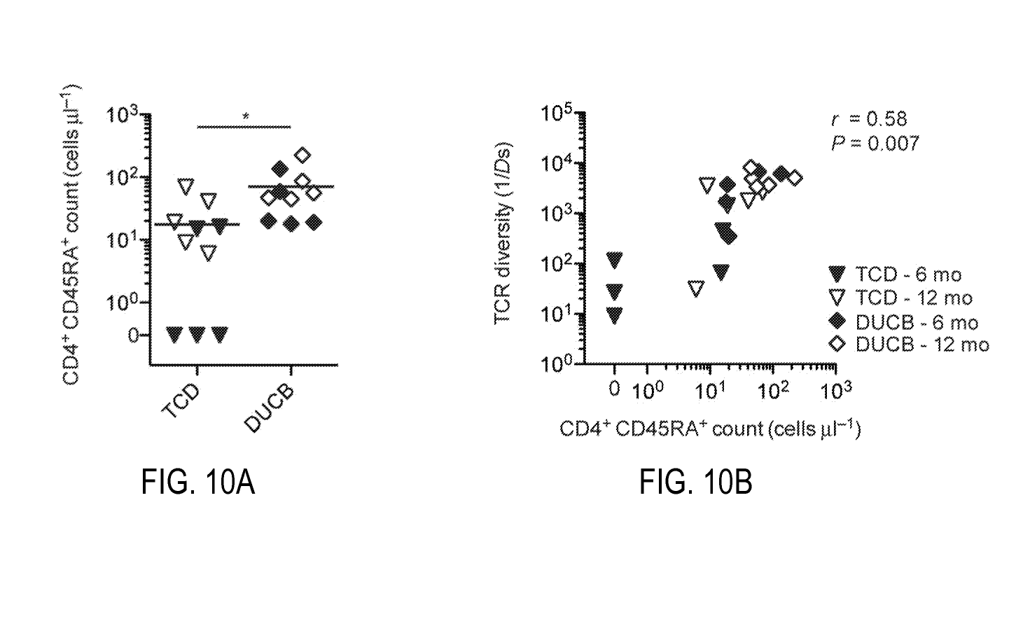

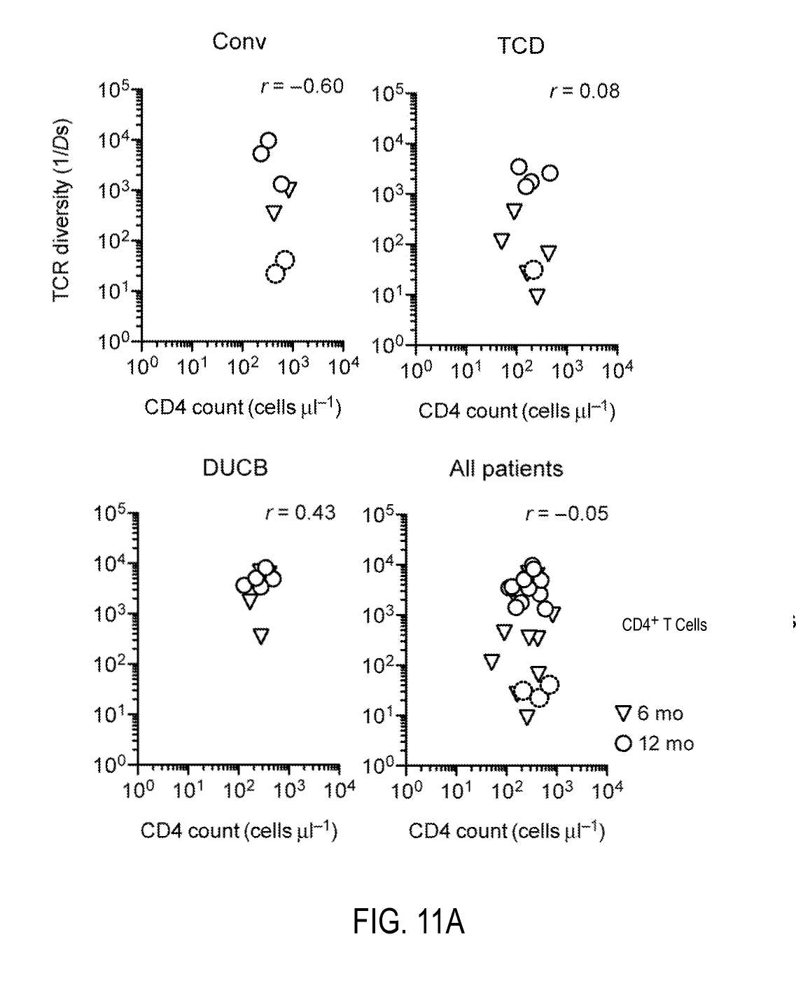

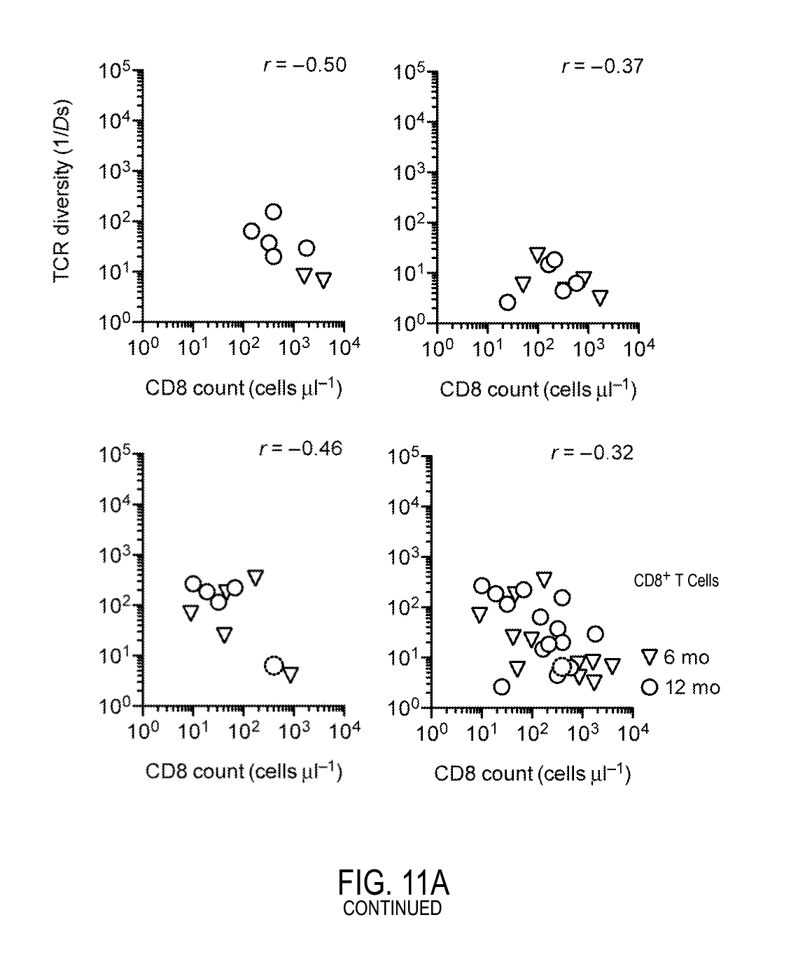

II) DUCB recipients had the highest TCR diversity of all patients and had a significantly (28-fold; P=0.033) more diverse CD4⁺ T-cell repertoire compared to TCD recipients after 6 months. Importantly, this increased TCR diversity also correlated with a substantially greater fraction of naïve CD4⁺ T cells in DUCB compared to TCD recipients (FIG. 10). Although TCD recipients had limited CD4⁺ T-cell diversity after 6 months, this diversity was 14-fold higher after 12 months, reducing the difference with DUCB recipients to 3-fold. III) Regarding CD8⁺ T cells, DUCB recipients again had the highest TCR diversity of all patients, which was 14-fold higher than TCD recipients after 6 months and 17-fold higher after 12 months, thereby reaching statistical significance (P=0.012).

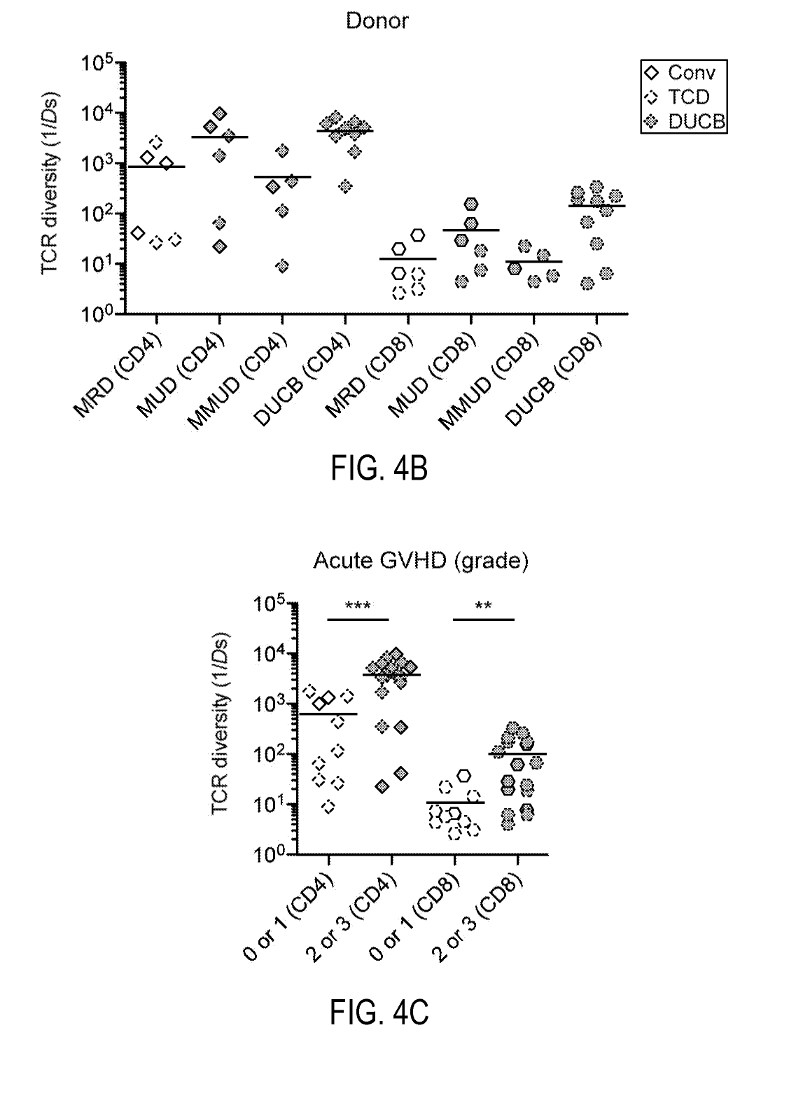

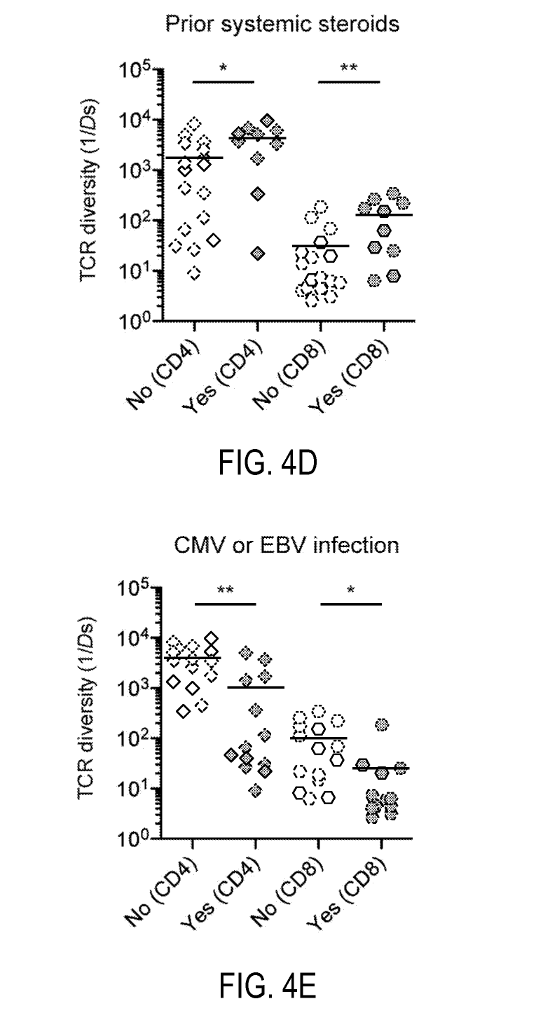

No significant impact of age or donor on TCR diversity (FIG. 4a,b). Interestingly, acute GVHD (grade 2 or 3) and systemic steroid treatment were associated with higher TCR diversity, suggesting that these variables do not restrict repertoire recovery (FIG. 4c,d). In contrast, cytomegalovirus (CMV) or EBV infections were associated with lower TCR diversity (FIG. 4e).

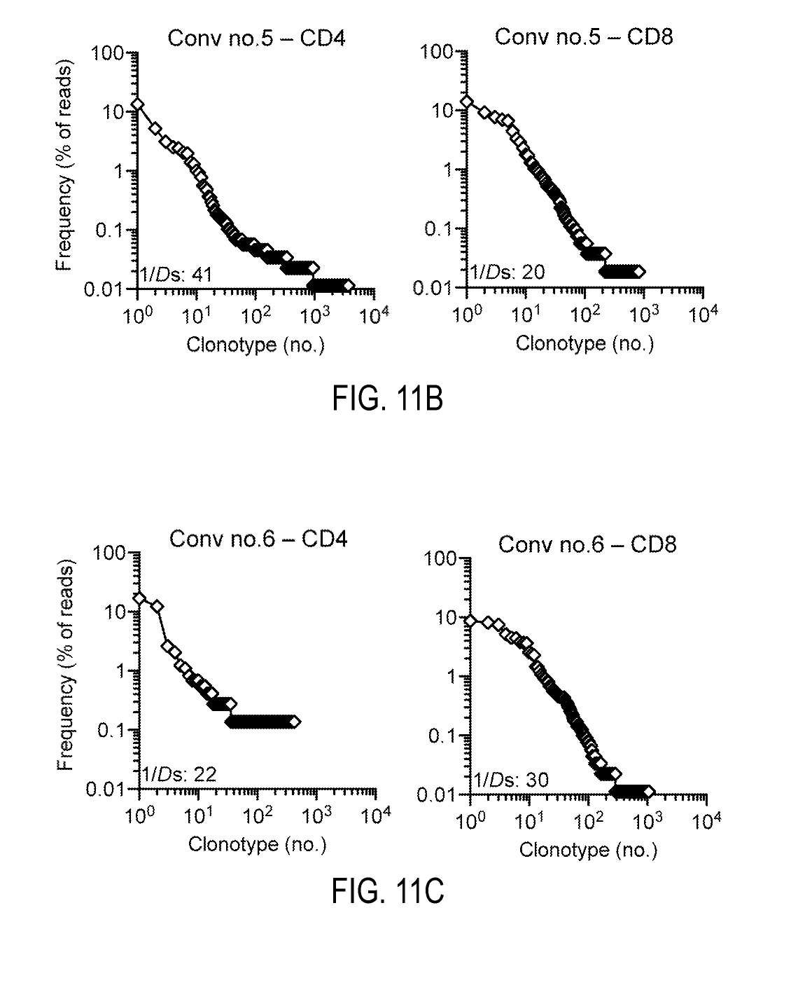

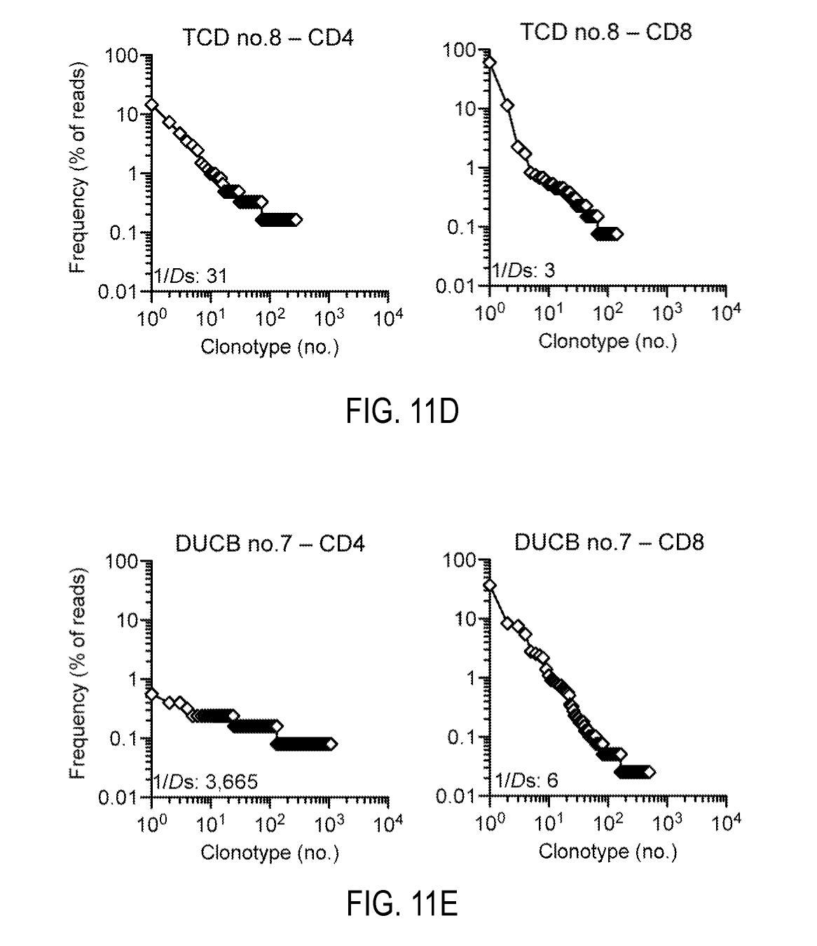

Within each stem cell group, patients were identified who had normal T-cell counts, but very low TCR diversity after one year (FIG. 11).

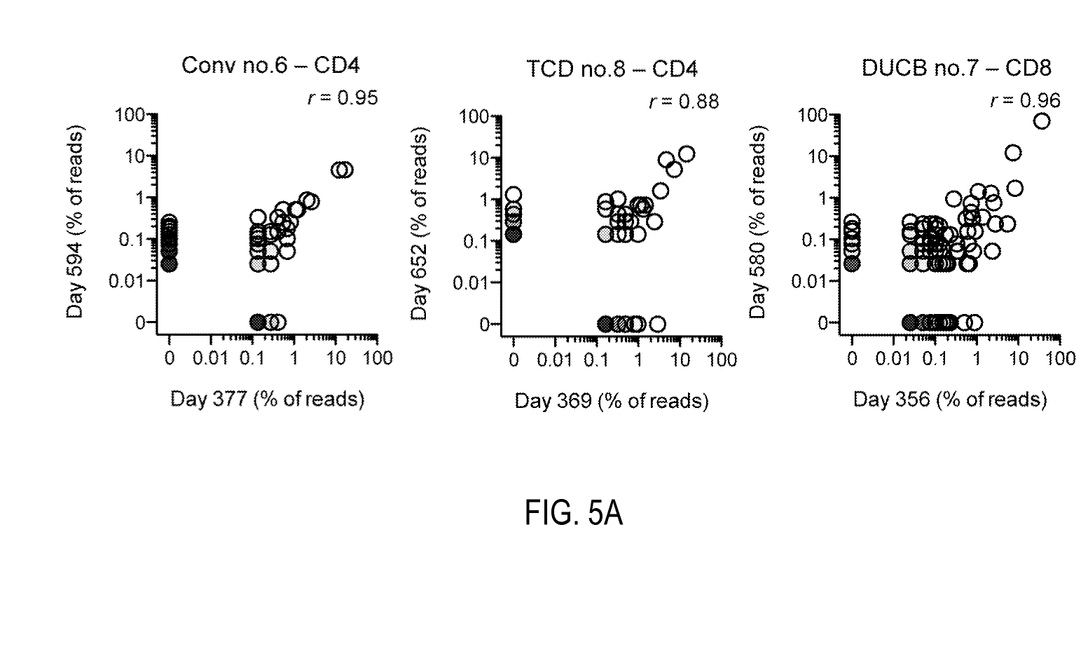

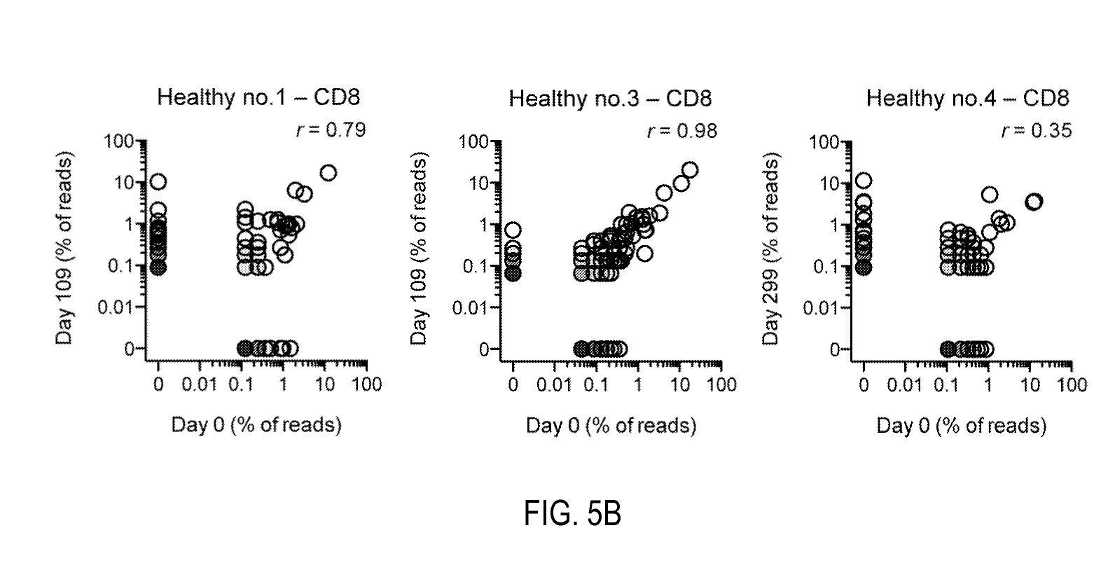

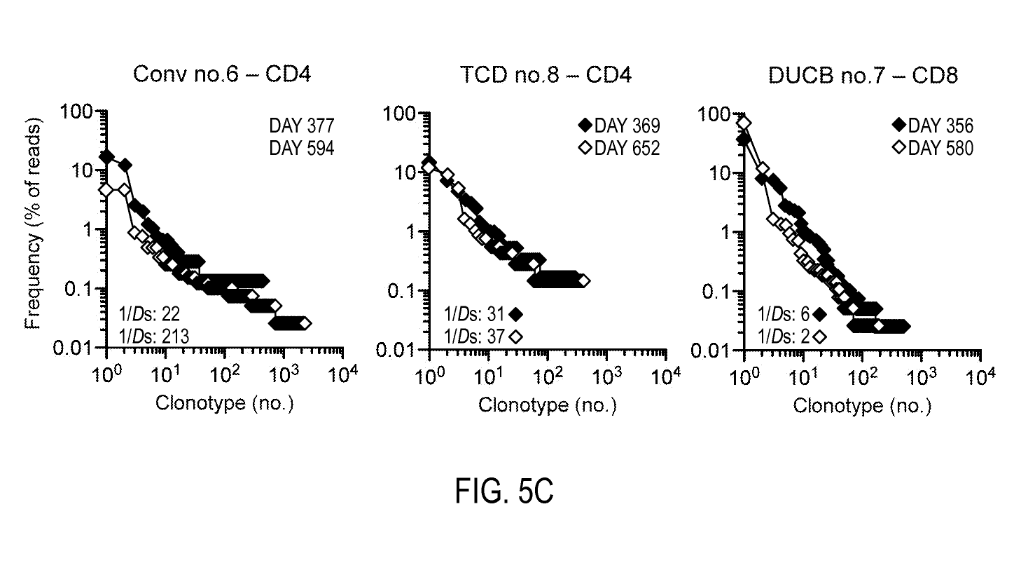

The CD4⁺ T-cell and CD8⁺ T-cell repertoires were stable over time (19-21 months) in of certain transplant patients [CD4⁺ T-cell: Conv #6 and TCD #8; and CD8⁺ T-cell: DUCB #7] as well as in healthy subjects. FIG. 5.

### EXAMPLES

### Example 1

### Methods

Patients.

28 patients with various hematologic malignancies underwent allo-HSCT at Memorial Sloan-Kettering Cancer Center (MSKCC) from April 2010 through September 2011. Patient and treatment characteristics are summarized in Table 1. Pre-transplant conditioning varied according to the patient's age, diagnosis, remission status, extent of prior therapies, and co-morbidities; and consisted of high-dose, reduced-intensity myeloablative and nonmyeloablative regimens³¹. GVHD prophylaxis for peripheral blood stem cell transplantation was either with T-cell depletion³² or calcineurin inhibitor-based, and ATG was used according to protocol or physician preference. Cord blood recipients received mycophenolate mofetil and calcineurin inhibitors; however, no patient received ATG³⁰. Post-transplant granulocyte colony-stimulating factor (G-CSF)³³ was used in all patients. Acute and late acute/chronic GVHD were diagnosed clinically with histological confirmation when possible. Staging of acute GVHD was graded according to CIBMTR criteria³⁴. Blood samples were obtained by venipuncture after written informed consent under MSKCC protocol 08-047.

T-Cell Isolation and Flow Cytometry.

From each ˜8 ml heparinized blood sample, mononuclear cells were isolated by Ficoll density centrifugation (Lymphocyte Separation Medium, MP Biomedicals). Recovered cells were lysed in RLT buffer (QIAGEN), homogenized using QIAshredder columns (QIAGEN) and stored at −80° C. until further use. For CD4⁺ and CD8⁺ T-cell separation, two ˜8 ml heparinized blood samples were pooled, followed by isolation of the mononuclear cell fraction as above. Recovered cells were split into two fractions and incubated with either human CD4 or CD8 MicroBeads (Miltenyi Biotec). CD4⁺ and CD8⁺ T cells were separated using MS columns (Miltenyi Biotec). Eluted cells were lysed, homogenized and stored as above. To determine the efficiency of T-cell separation, eluted cells were stained with FITC anti-human CD14 (clone M5E2), PE-Cy7 anti-human CD4 (clone SK3) and APC anti-human CD8 (clone RPA-T8; all BD Pharmingen); and measured on an LSRII flow cytometer (BD Biosciences). Data was analyzed using FlowJo software (TreeStar). For separation of naïve and memory CD8⁺ T-cells, isolated mononuclear cells were stained with FITC anti-human CD45RA (clone HI100), PE anti-human CD45RO (clone UCHL1; both BD Pharmingen) and APC anti-human CD8. Cells were sorted using a FACSAria cell sorter (BD Biosciences) into CD8⁺CD45RA⁺CD45RO⁻ (naïve) and CD8⁺CD45RA⁻CD45RO⁺ (memory) fractions.

5′-Race PCR and Roche/454 Sequencing.

Total RNA from frozen homogenates was extracted using an RNeasy mini kit (QIAGEN). RACE-Ready cDNA was generated using a SMARTer RACE cDNA Amplification kit (Clontech) and oligo(dT) or random (N-15) primers. 5′-RACE PCR was performed using Advantage 2 Polymerase mix (Clontech) with Clontech's universal forward primer and a self-designed universal TCRβ-constant reverse primer compatible with both human TRBC gene segments (5′-GCACACCAGTGTGGCCTTTTGGG-3′ SEQ ID NO. 6). Amplification was performed on a Mastercycler pro (Eppendorf) and was 1 min at 95° C.; 5 cycles of 20 sec at 95° C. and 30 sec at 72° C.; 5 cycles of 20 sec at 95° C., 30 sec at 70° C. and 30 sec at 72° C.; 25 cycles of 20 sec at 95° C., 30 sec at 60° C. and 30 sec at 72° C.; 7 min at 72° C. PCR products were loaded on 1.2% agarose gels (Bio-Rad) and bands centered at ˜600 bp were excised and purified using a MinElute Gel Extraction kit (QIAGEN). Purified products were subjected to a second round of amplification to introduce adaptor sequences compatible with unidirectional Roche/454 sequencing. 1/50^{th} of first-round PCR product was amplified using Advantage 2 Polymerase mix with a hybrid forward primer consisting of Roche's Lib-L primer B and Clontech's nested universal primer (5′-CCTATCCCCTGTGTGCCTTGGCAGTCTCAGAAGCAGTGGTATCAACGCAGAGT-3 SEQ ID NO. 7′) and a hybrid reverse primer consisting of Roche's Lib-L primer A and a self-designed nested universal TCRβ-constant primer (5′-CCATCTCATCCCTGCGTGTCTCCGACTCAG-MID-AACACAGCGACCTCGGGTGGGAA-3′ SEQ ID NO. 8), wherein MID represents the multiplex identifier used to separate pooled samples during sequence analysis). The multiplex identifier is essentially a bar code that is added to primers so that multiple samples can be resolved after high throughput sequences of a mixture of samples. Multiplex identifiers were 6-7 bp long. Amplification was 1 min at 95° C.; 25 cycles of 20 sec at 95° C., 30 sec at 68° C. and 30 sec at 72° C.; 7 min at 72° C. PCR products were purified from agarose gels as above. Purified products were measured, pooled and sequenced using the GS Junior 454 platform (Roche) following the manufacturer's instructions.

Sequence Data Analysis.

Raw sequence data was converted to FASTA format using MOTHUR software³⁵. Sequences shorted than 125 bp, with uncalled bases, with a Phred quality score average below 30 (base call accuracy <99.9%)²⁷, or with no exact match to the TCRβ-constant primer or a multiplex identifier were discarded. Resulting FASTA files were uploaded to the IMGT/HighV-QUEST database (http://www.imgt.org/HighV-QUEST/index.action)³⁶. Using IMGT summary files, sequences with out-of-frame rearrangements, with a V- and J-region identity <80%, with V-region pseudogenes or a CDR3β AA junction lacking a 5′ cysteine and 3′ phenylalanine were discarded. Resulting sequences were sorted using Excel (Microsoft) and graphed using Prism 5 software (GraphPad). The inverse Simpson's diversity index (1/Ds) was calculated using MOTHUR.

Statistical Analysis.

TCR diversity was compared using an unpaired Student's t-test (two groups) or one-way ANOVA with Bonferroni's multiple comparison test (three groups). A P-value of <0.05 was considered statistically significant.

### Example 2

### Strategy and Reproducibility of T-Cell Repertoire Analysis

T cells typically express only one productively recombined TCRβ chain, making sequence analysis of TCRβ cDNA a useful measure of T-cell repertoire complexity. To evaluate the human TCRβ repertoire in a faithful manner, use was mad of 5′ rapid amplification of cDNA ends (RACE), which allows amplification of all 48 different Vβ genes using a single oligonucleotide pair. Other amplification methods can be used in the methods of the invention.

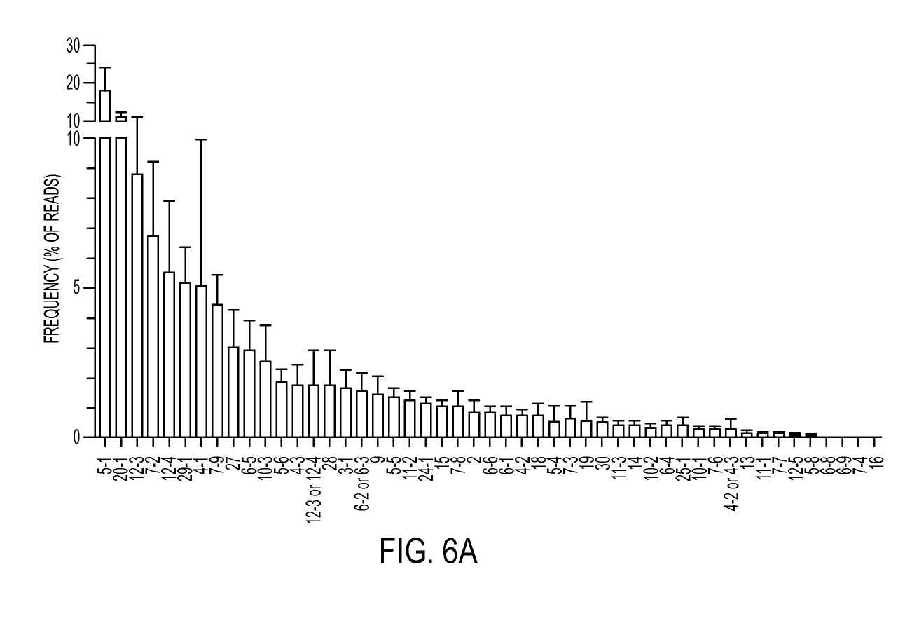

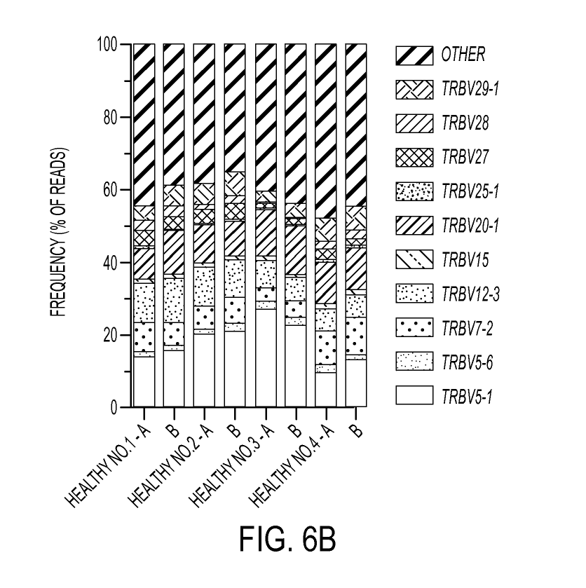

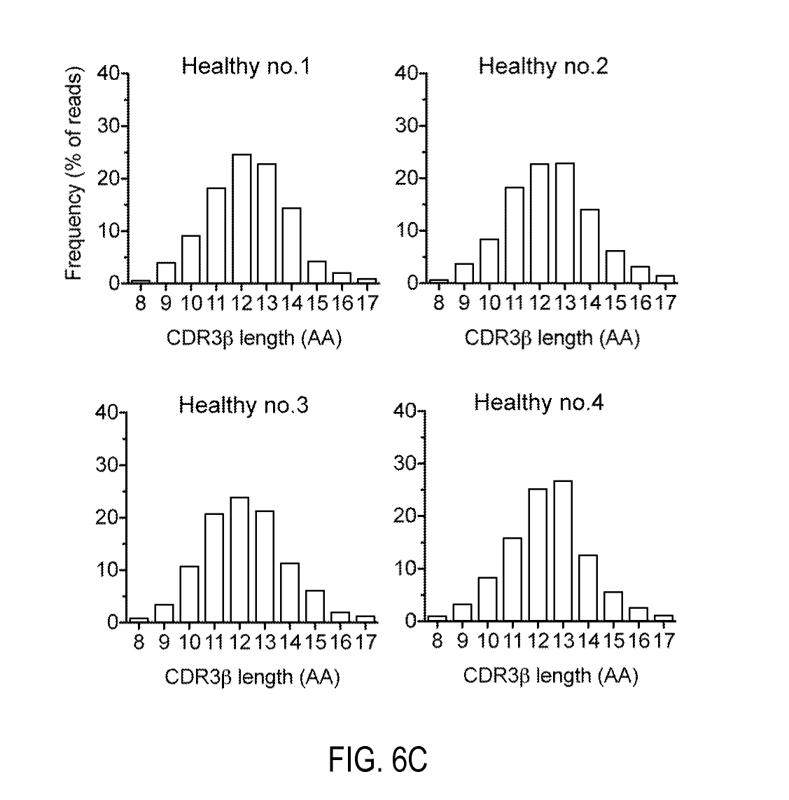

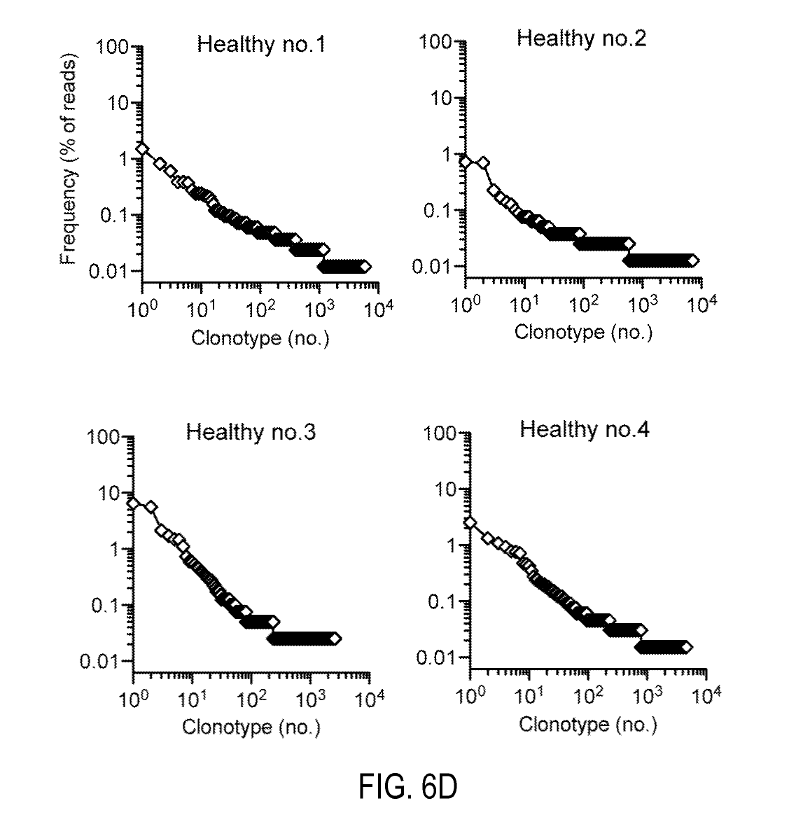

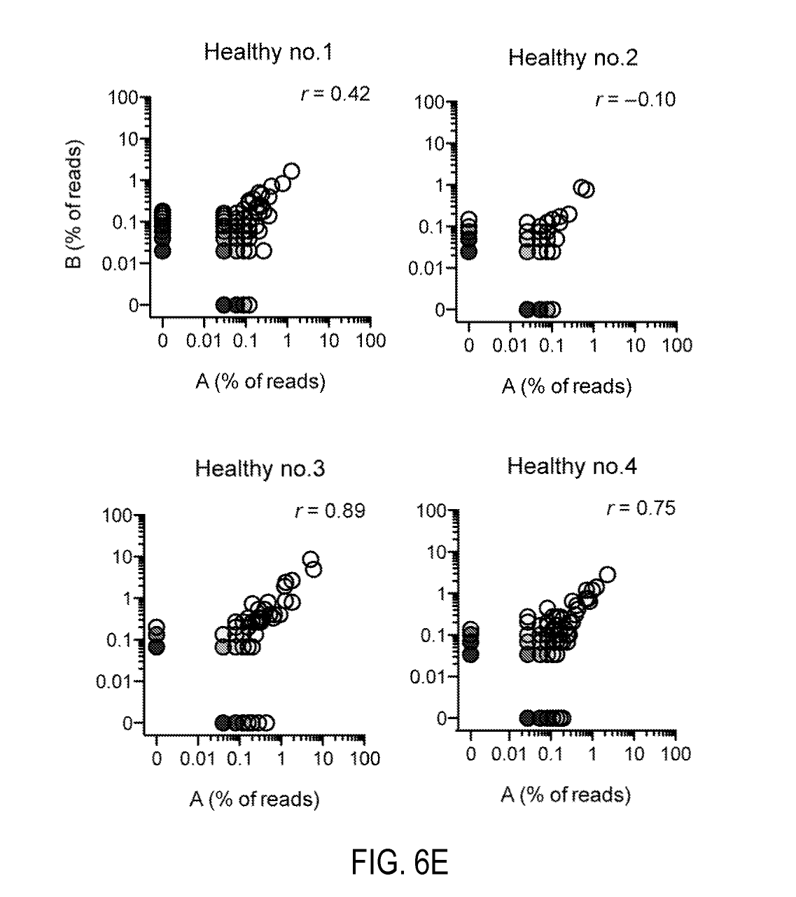

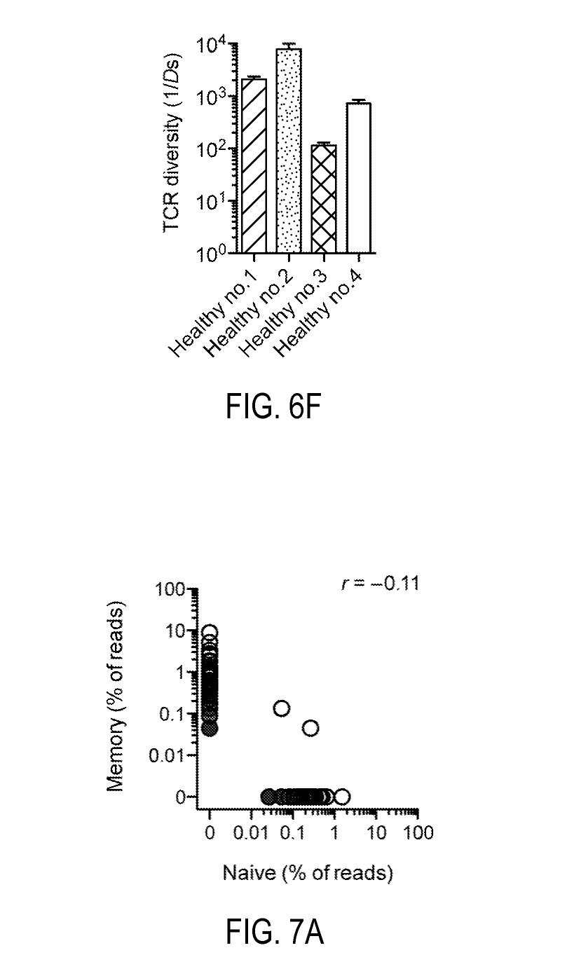

To test the reproducibility of this approach, separately amplified were two ˜8 ml blood samples that were obtained from a single patient TCD #1, after written informed consent, 138 days following T-cell-depleted (TCD) peripheral blood stem cell transplantation (TCD #1; Table 1). Since the T-cell repertoire of this patient was likely severely restricted, also separately amplified were two blood samples from each of four healthy subjects (Healthy #1-4; Table 1), to serve as a reference for normal high TCR diversity. Following amplification, all samples were analyzed by Roche/454 sequencing or Illumina MiSeq, using a Phred quality score average of 30 to minimize the contribution of sequence errors²⁷. Among total TCR sequences, all 48 different Vβ genes were found in the subjects_(FIG. 6), indicating that the present approach allows coverage of the entire human TCRβ repertoire. Furthermore, comparison of the two patient blood samples from TCD #1 showed a highly reproducible pattern of TCR Vβ usage, which differed markedly from the four healthy subjects (FIG. 1a). Specifically, Vβ15 was used by 29.6% of TCRs in TCD #1, whereas it was only used by 1.1±0.1% of TCRs in the four healthy subjects. This suggested that the repertoire of TCD #1 contained substantial clonal expansions compared to Healthy #1-#4, perhaps reflecting viral infection or the development of graft-versus-host-disease. To investigate this further, digital CDR3 size spectratype profiles were generated using all TCR sequences, which revealed a prominent over-representation of TCRs with a CDR3β length of 11 amino acids in TCD #1 (FIG. 1b).

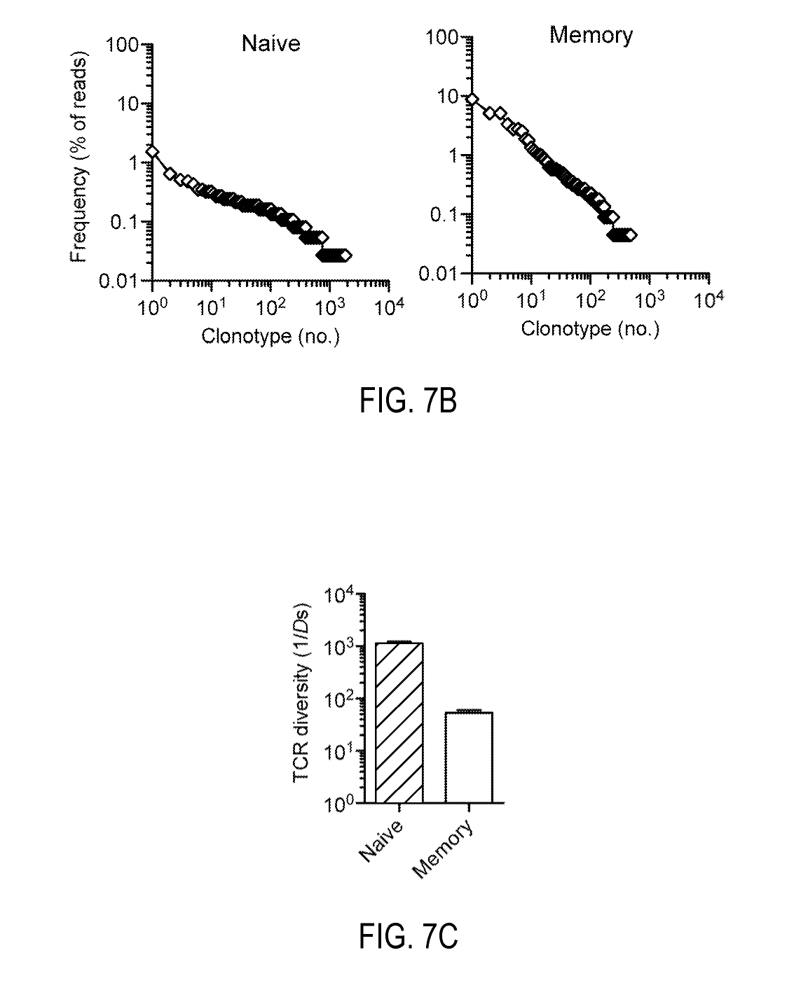

Next, the frequency was determined at which each distinct TCRβ chain, or TCRβ clonotype, was present among the total pool of TCRs. In 15,902 reads obtained from both blood samples of the TCD #1, 1,097 distinct TCRβ clonotypes, were detected with the most frequent clonotype comprising 11.7% of the total repertoire (FIG. 1c). In fact, 19 clonotypes that were present at a frequency of 1% or more were found and together constituted 70.8% of all reads, indicating that the TCD #1 repertoire contained several highly abundant TCRs. In contrast, the most abundant clonotype in the four healthy controls comprised just 2.8±2.6% of the repertoire, and on average only 3 clonotypes were found above 1% (FIG. 1c and FIG. 7c).

To establish how accurately the frequency of individual clonotypes had been determined, the clonotype distribution of both blood samples from TCD #1 were compared. Importantly, abundant clonotypes found in TCD #1 blood sample were found with almost identical frequency in the second blood sample (also from TCD #1), resulting in a near-perfect correlation of both clonotype distributions (r: 0.98; FIG. 1d). In the four healthy subjects, expanded clonotypes were also detected with high reproducibility, however the average correlation between two blood samples was lower (r: 0.44; FIG. 6), primarily because fewer clonotypes passed the abundance threshold for physical presence in a second blood sample (8 ml out of ˜5 liter total=˜0.16%).

To provide a quantitative value for TCR diversity, the inverse Simpson's diversity index (1/Ds) was used, which sums the fraction each clonotype makes up of the total repertoire²⁸. This index ranges from 1 (no diversity) to ∞ (infinite diversity) and is highest when all clonotypes have an equal distribution. To test the usefulness of this index, naïve and memory CD8⁺ T cells from Healthy #1 were sorted and a 20-fold higher TCR diversity found in the naïve T-cell compartment compared to the memory CD8⁺ cells (FIG. 7). Analysis of the TCD #1 repertoire revealed a very low TCR diversity (1/Ds: 23), which was more than 100-fold lower than the average diversity of four healthy subjects (1/Ds: 2,525; FIG. 1e and FIG. 7e). Therefore, at 138 days after transplant the TCD #1 patient had a poorly recovered T-cell repertoire.

### Example 3

### Dynamics of T-Cell Repertoire Recovery after Allo-HSCT

Previous studies have suggested a considerable lag time in the restoration of thymic activity after allo-HSCT^{8,10}. Based the observation that individual clonotypes can comprise as much as 10% of the repertoire 4.5 months after transplant, it is speculated that over time such clonotypes should be diluted out by the addition of recent thymic emigrants, driving the repertoire towards a more even distribution⁹.

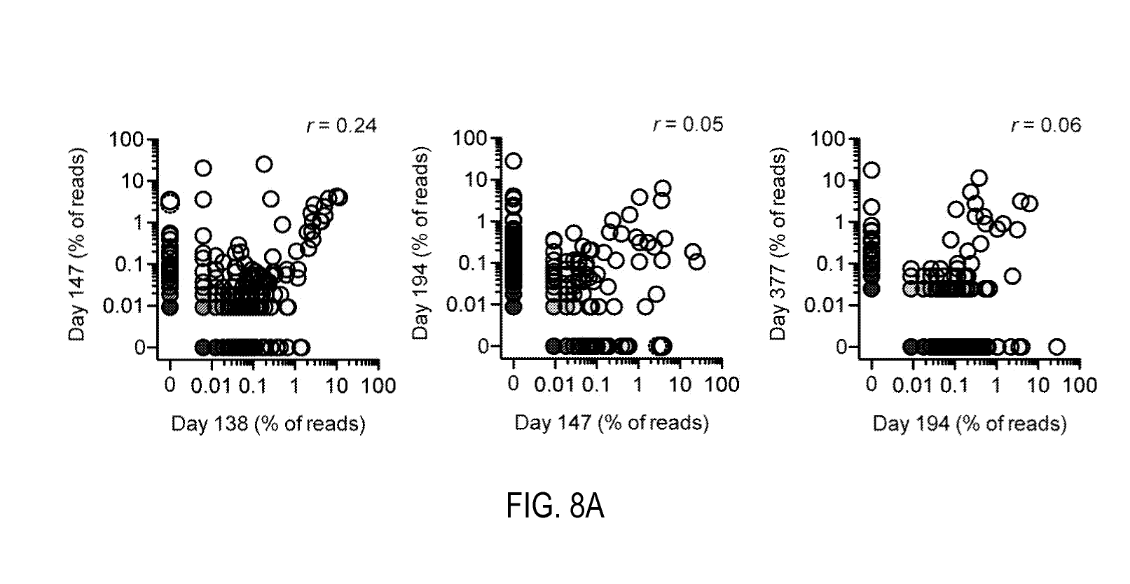

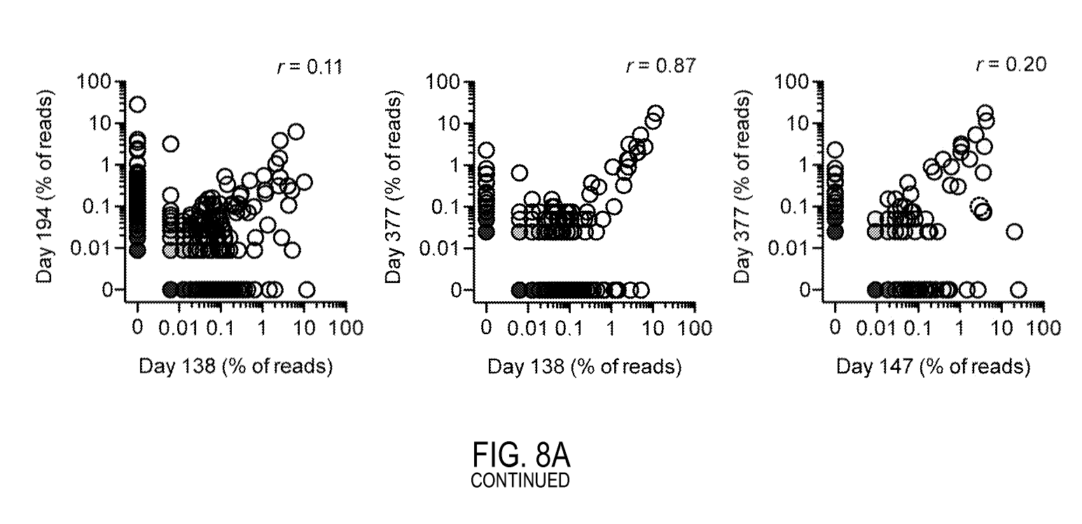

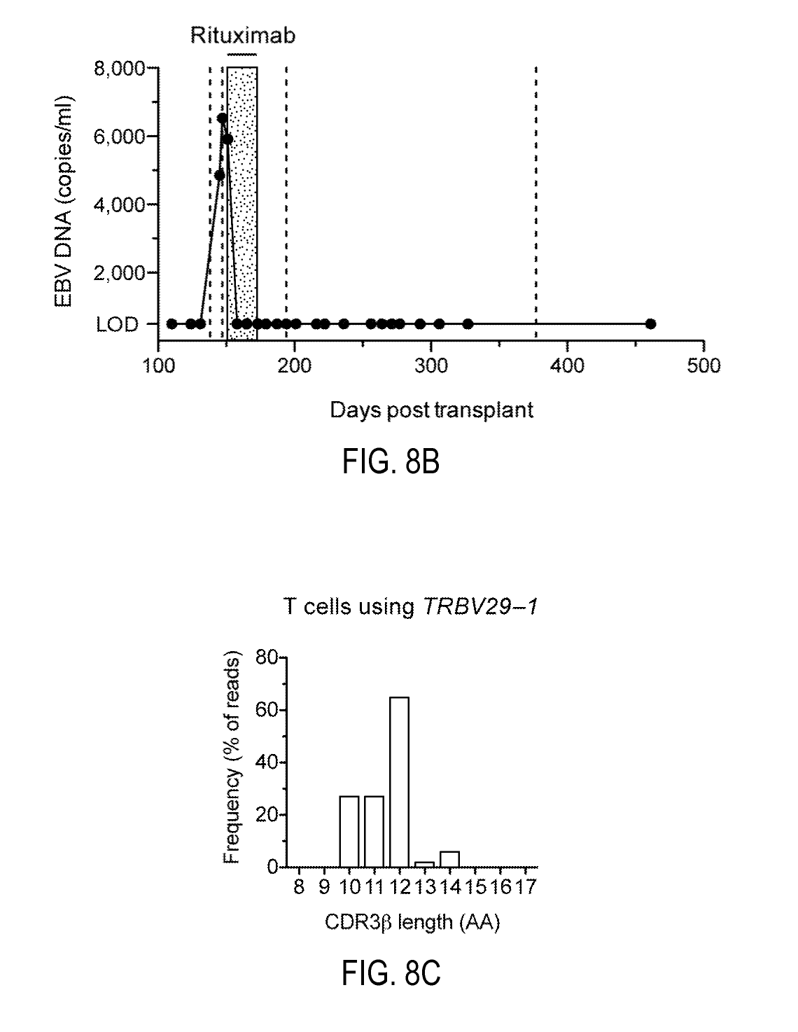

To monitor repertoire recovery over time, three additional timepoints of TCD no. 1 were measured at days 147, 194 and 377 after transplant. To our surprise, TCR Vβ usage was very different at each timepoint examined (FIG. 2a), indicating high variability of the T-cell repertoire. To evaluate individual clonotypes, it was first determined whether clonotype frequencies were reliably measured. On day 147, it was again found a near-perfect correlation between two blood samples (r: 0.99), and the same held true for days 194 and 377 (r: 1 and 0.98, respectively; FIG. 2b). Comparison of the repertoire on days 138 and 147, however, revealed dramatic shifts in clonotype frequencies (FIG. 2c). Although some clonotypes were present at roughly similar frequency on both days, several others differed by more than 100-fold; resulting in a low similarity between both T-cell repertoires measured just 9 days apart (r: 0.24). Importantly, these repertoire shifts coincided with Epstein-Barr virus (EBV) reactivation in the patient, which was first detected on day 145 and peaked on day 147 (FIG. 8). Identified was the 9^{th} most abundant clonotype on day 147 (TRBV29-1, CDR3β CSVGTGGTNEKLFF/SEQ ID NO. 1) as being specific for the HLA-A2-restricted BMLF1₂₈₀ epitope from EBV³². While undetectable on days 138 and 194, this clonotype comprised 2.9% of reads on day 147 and 0.1% on day 377, highlighting the potential of our method to track antigen-specific clonotypes. Despite apparent resolution of EBV reactivation by day 158, subsequent blood samples continued to reveal clonotype frequencies fluctuating over orders of magnitude. Thus, the repertoire of two samples on the same day was highly similar (r: 0.99), whereas on different days it was markedly distinct (r: 0.25; FIG. 2d). A notable exception was the repertoire of days 138 and 377, which revealed a surprisingly large degree of similarity (r: 0.87; FIG. 8). Despite profound changes in repertoire composition, TCR diversity did not increase over time (1/Ds: 23 and 19 for days 138 and 377, respectively; FIG. 2e). Therefore, between 4.5 and 12.5 months after transplant, the complexity of this patient's T-cell repertoire did not improve.

### Example 4

### T-Cell Repertoire Recovery by Different Stem Cell Sources Stratification of Allo-HSCT Recipients According to their TCR Diversity

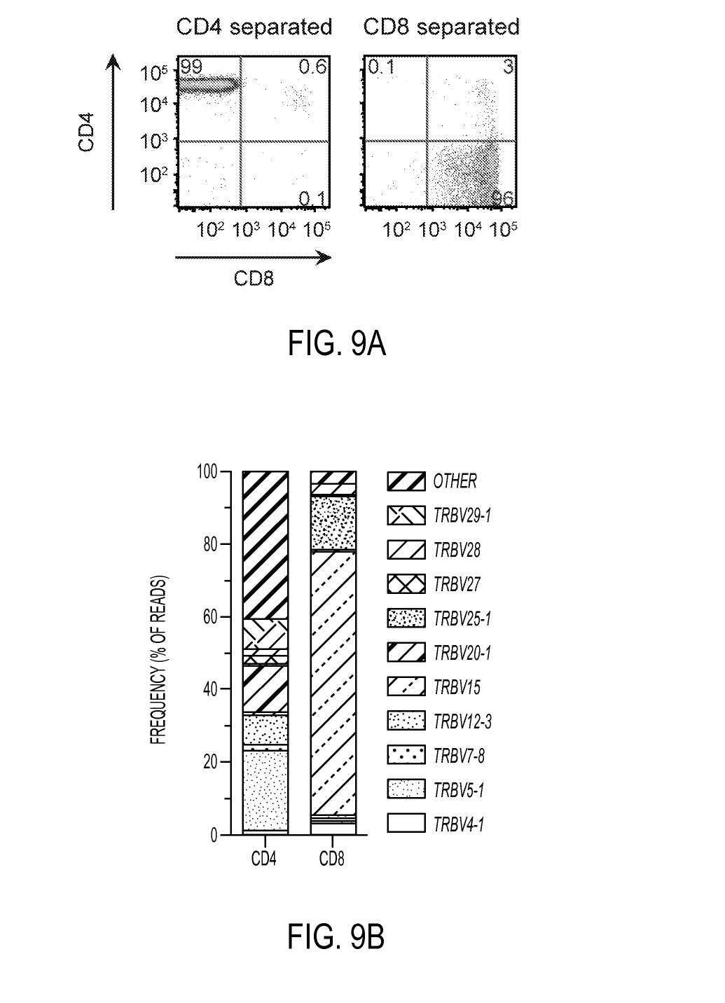

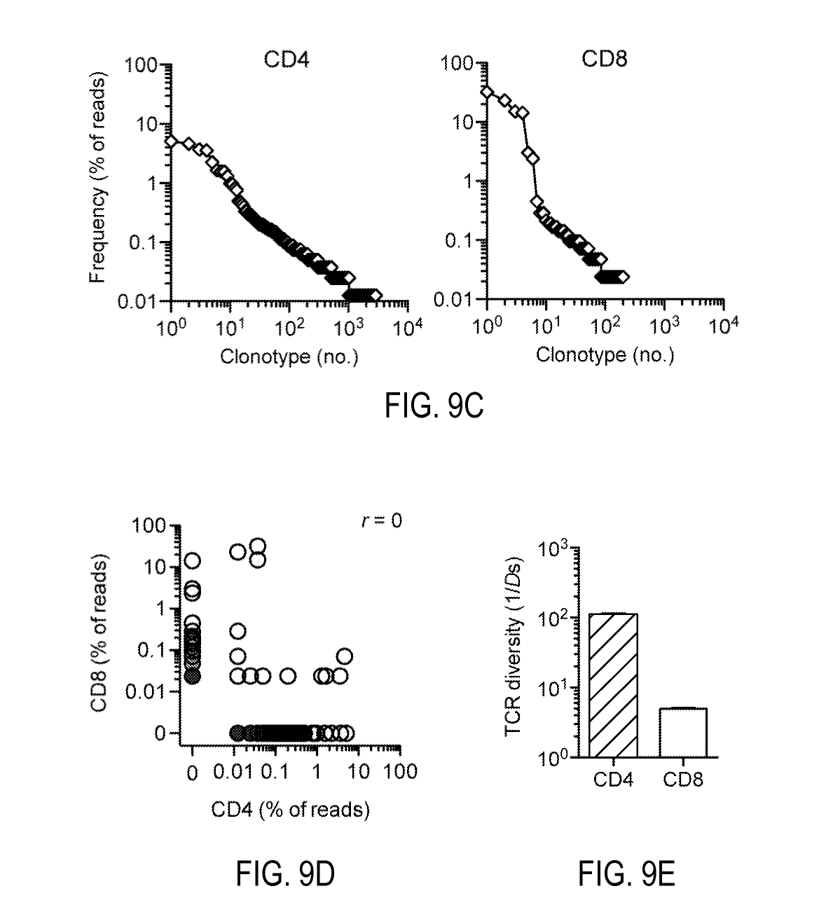

Given that the presented method allows accurate characterization of human T-cell repertoires, a method was developed to stratify allo-HSCT recipients according to their TCR diversity, thereby enabling identification of patients with particularly narrow T-cell repertoires. As a first step, TCR diversity was measured in recipients of three different stem cell sources at two different time points²⁵. To this end, 27 cancer patients who received transplants were sequenced at either 6 or 12 months after either conventional (Conv) or T-cell-depleted (TCD) peripheral blood stem cell transplantation, or double-unit umbilical cord blood (DUCB) transplantation without anti-thymocyte globulin³⁰ (Table 1, FIG. 3a-c).). Because analysis of TCD #1 had suggested substantially greater TCR diversity in CD4⁺ T cells compared to CD8⁺ T cells (FIG. 10), both T-cell compartments were separately analyzed for all 27 patients and healthy subjects In addition, the CD4⁺ and CD8⁺ T-cell repertoires of five age-matched healthy subjects were sequenced (Table 1, FIG. 9).

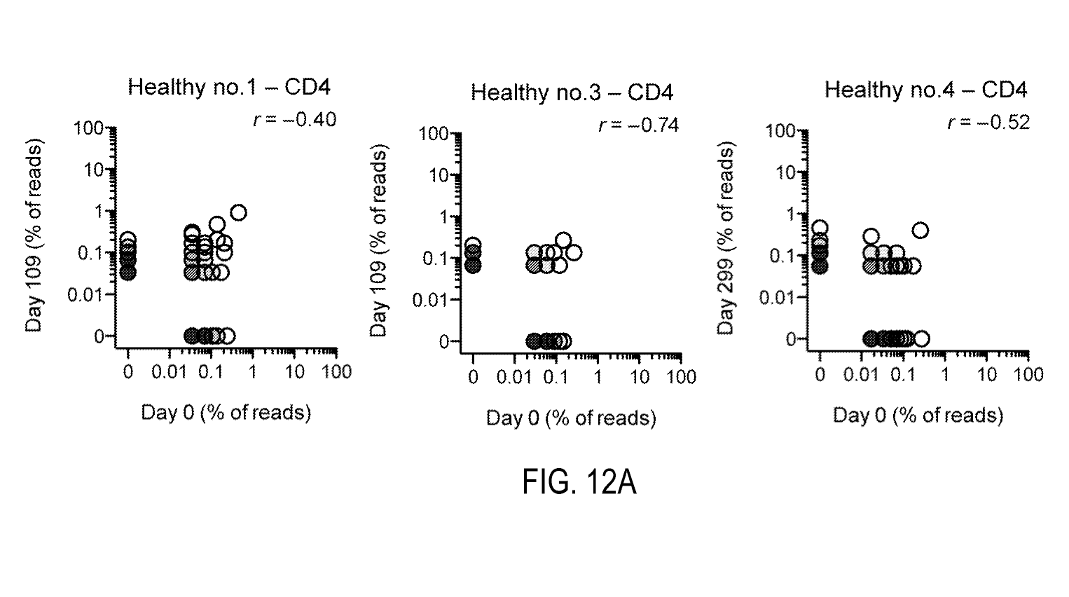

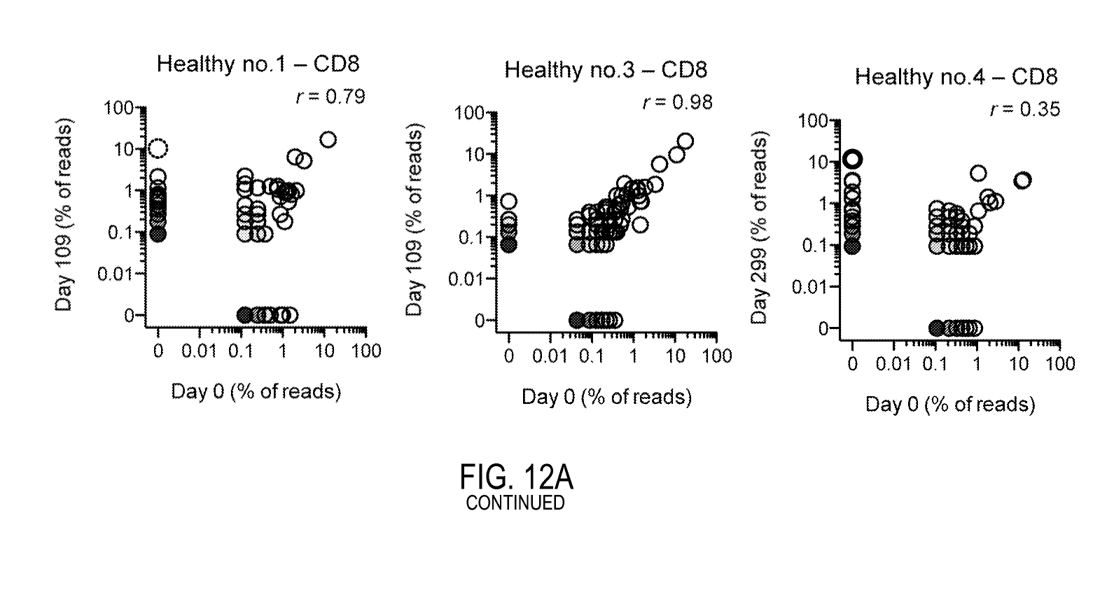

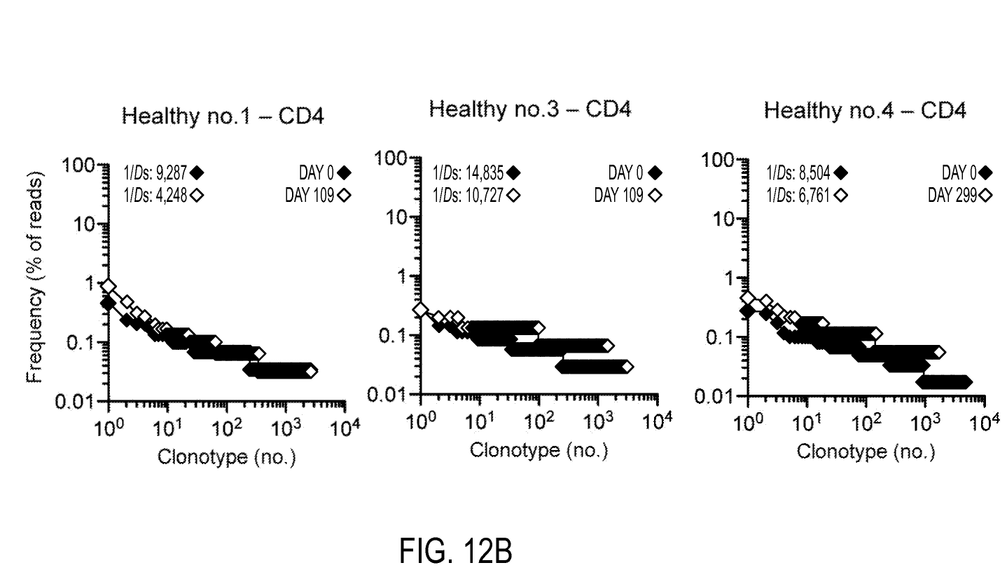

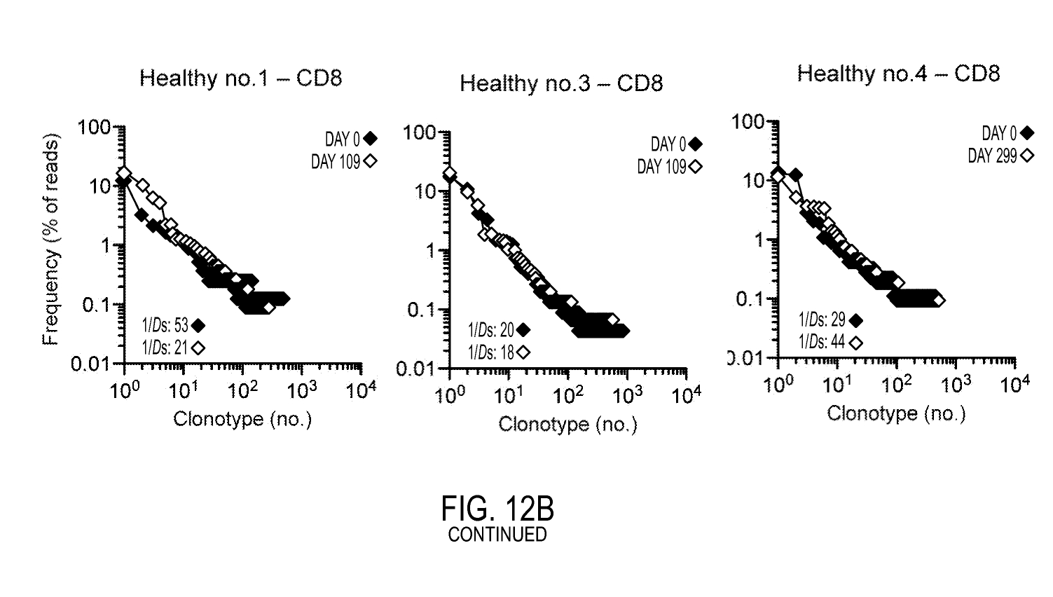

Analysis of total TCR sequences indicated that Vβ gene usage between both T-cell compartments followed markedly distinct patterns (FIG. 11). While in CD4⁺ T cells the pattern of Vβ selection was generally conserved between individuals as well as between different grafts, in CD8⁺ T cells this selection showed much greater variability. Nevertheless, there was a clear hierarchy in Vβ usage among total T cells, ranging from Vβ5.1 (15.9±11.5%) to Vβ6.9 (0.008±0.024%; FIG. 12).

### The Clonotype Distribution of the Individual CD4⁺ and CD8⁺ T-Cell Repertoires

Next, the clonotype distribution of the individual CD4⁺ and CD8⁺ T-cell repertoires were determined for the 27 transplant patients. FIG. 3 shows a representative example of transplant recipient after either 6 or 12 months, as well as a representative healthy individuals.

After determining the TCR diversity of each individual, it was established that: I) for all stem cell sources as well as for healthy donors, CD4⁺ T-cell diversity was ˜50-times higher than CD8⁺ T-cell diversity (1/Ds: 4,665 and 81, respectively; FIG. 3e,f). II) Regarding CD4⁺ T cells, healthy donors had the highest TCR diversity (1/Ds: 15,470), followed by DUCB after 12 months (1/Ds: 5,069), DUCB after 6 months (1/Ds: 3,745), Conv after 12 months (1/Ds: 3,298), TCD after 12 months (1/Ds: 1,871), Conv after 6 months (1/Ds: 674) and TCD after 6 months (1/Ds: 132). Therefore, DUCB recipients had the highest TCR diversity of all patients and had a significantly (28-fold; P=0.033) more diverse CD4⁺ T-cell repertoire compared to TCD recipients after 6 months. Importantly, this increased TCR diversity also correlated with a substantially greater fraction of naïve CD4⁺ T cells in DUCB compared to TCD recipients (FIG. 10). Although TCD recipients had limited CD4⁺ T-cell diversity after 6 months, this diversity was 14-fold higher after 12 months, reducing the difference with DUCB recipients to 3-fold. III) Regarding CD8⁺ T cells, DUCB recipients again had the highest TCR diversity of all patients, which was 14-fold higher than TCD recipients after 6 months and 17-fold higher after 12 months, thereby reaching statistical significance (P=0.012).

### Example 5

### T-Cell Repertoire Recovery and Clinical Variables

Using above data, several clinical parameters were investigated that could influence T-cell repertoire recovery. No significant impact was found of age or donor on TCR diversity (FIG. 4a,b). Interestingly, acute GVHD (grade 2 or 3) and systemic steroid treatment were associated with higher TCR diversity, suggesting that these variables do not restrict repertoire recovery (FIG. 4c,d). In contrast, cytomegalovirus (CMV) or EBV infection were associated with lower TCR diversity (FIG. 4e).

### Example 6

### Monitoring Patients with Poor T-Cell Repertoire Recovery

Using above data, several clinical parameters were investigated that could influence T-cell repertoire recovery. No significant impact was found of age or donor on TCR diversity (FIG. 4a,b). Interestingly, acute GVHD (grade 2 or 3) and systemic steroid treatment were associated with higher TCR diversity, suggesting that these variables do not restrict repertoire recovery (FIG. 4c,d). In contrast, cytomegalovirus (CMV) or EBV infection were associated with lower TCR diversity (FIG. 4e).

Within each stem cell group, patients were identified who had normal T-cell counts, but very low TCR diversity after one year (Supplementary FIG. 6). To investigate repertoire recovery during the second year of transplant, TCR diversity was reanalyzed in three of these patients after 19-21 months. For comparison, three healthy donors were also reanalyzed. Stability was found in the CD4⁺ T-cell repertoires of Conv no. 6 and TCD no. 8, and the CD8⁺ T-cell repertoire of DUCB no. 7 (FIG. 5a). Similar stability was observed in the CD8⁺ T-cell repertoires of healthy donors when measured over 109 days, whereas over 299 days there was somewhat greater divergence (FIG. 5b). Despite occasional changes in clonotype frequencies, TCR diversity in healthy donors was stable (FIG. 12). Interestingly, in Conv no. 6 the frequency of abundant clonotypes had substantially decreased over time, resulting in a 10-fold higher CD4⁺ T-cell diversity (FIG. 5c). In contrast, there was no diversification in the other two patients. Together, these data illustrate the potential of our method to identify patients as well as transplant protocols that are associated with either greater or lesser T-cell repertoire recovery.

### Discussion

In this study, a methodology is established to measure human TCR diversity in a reproducible and quantitative way. By combining 5′-RACE PCR of TCRβ cDNA with deep sequencing, this method allows assessment of the entire human TCRβ repertoire using a single oligonucleotide pair for amplification. This method was validated by measuring T-cell repertoire recovery in allo-HSCT recipients, in whom limited TCR diversity is linked to susceptibility to infection and cancer relapse. Overall, significant improvement was found in TCR diversity between 6 and 12 months after transplant, providing proof-of-principle that the presented method can document T-cell repertoire recovery. However, there was substantial variability in the rate of repertoire recovery by different stem cell sources. Interestingly, cord blood recipients demonstrated superior TCR diversity over peripheral blood stem cell recipients, and approximated the TCR diversity of healthy subjects by 6 months. It is important to note that all cord blood transplantations are performed without the inclusion of anti-thymocyte globulin (ATG) in the preparative regimen, and this has recently been shown to be associated with a ˜3.5-fold faster T-cell recovery after 6 months compared to ATG-based cord blood transplantation³⁰. Next to differences in transplant conditions, also identified were individual patients that had normal T-cell counts after 12 months, but 25- to 150-fold lower TCR diversity compared to their group mean. After 18 months, TCR diversity of one of these patients had improved substantially but that of others had not, illustrating the use of this method to gauge an individual patient's immunocompetence.

Although applied here in the setting of T-cell reconstitution after allo-HSCT, this method can be used to determine TCR diversity in a variety of immune diseases, such as HIV/AIDS and autoimmunity. Ultimately, the ability to monitor T-cell repertoire recovery with great precision provides new methods to identify agents that increase T cell repertoire recovery and immune regeneration.

Nucleic Acid Sequences:
Vb 29.1/CDR3b
SEQ ID NO. 1
CSVGTGGTNEKLFF
cDNA sequence:
(SEQ ID NO: 2)
GCCTTTTCTCAGGGGAGAGGCCATCACTTGAAGATGCTGAGTCTTCTGCT
CCTTCTCCTGGGACTAGGCTCTGTGTTCAGTGCTGTCATCTCTCAAAAGC
CAAGCAGGGATATCTGTCAACGTGGAACCTCCCTGACGATCCAGTGTCAA
GTCGATAGCCAAGTCACCATGATGTTCTGGTACCGTCAGCAACCTGGACA
GAGCCTGACACTGATCGCAACTGCAAATCAGGGCTCTGAGGCCACATATG
AGAGTGGATTTGTCATTGACAAGTTTCCCATCAGCCGCCCAAACCTAACA
TTCTCAACTCTGACTGTGAGCAACATGAGCCCTGAAGACAGCAGCATATA
TCTCTGCAGCGTTGGGACAGGAGGAACTAATGAAAAACTGTTTTTTGGCA
GTGGAACCCAGCTCTCTGTCTTGGAGGACCTGAACAAGGTG
AA sequence:
(SEQ ID NO: 3)
AFSQGRGHHLKMLSLLLLLLGLGSVFSAVISQKPSRDICQRGTSLTIQCQ
VDSQVTMMFWYRQQPGQSLTLIATANQGSEATYESGFVIDKFPISRPNLT
FSTLTVSNMSPEDSSIYLCSVGTGGTNEKLFFGSGTQLSVLEDLNKV

### Primers Used for PCR Amplification of TCR Genes

First round of PCR amplification

Forward primers:
(SEQ ID NO: 4)
5′CTAATACGACTCACTATAGGGCAAGCAGTGGTATCAACGCAGAGT3′
and
(SEQ ID NO: 5)
5′CTAATACGACTCACTATAGGGC3′
Reverse primer:
(SEQ ID NO: 6)
5′GCACACCAGTGTGGCCTTTTGGG3′
Second round of PCR amplification
Forward primer:
(SEQ ID NO: 7)
5′CCTATCCCCTGTGTGCCTTGGCAGTCTCAGAAGCAGTGGTATCAAC
GCAGAGT3′
Reverse primer:
(SEQ ID NO: 8)
5′CCATCTCATCCCTGCGTGTCTCCGACTCAG-MID-
AACACAGCGACCTCGGGTGGGAA3′

MID represents the multiplex identifier used to separate pooled samples during sequence analysis. Multiplex identifiers were 6-7 bp long.

### List of Used MID Sequences:

| | | | |
|---|---|---|---|
| A2 | CGCAAC | B1 | AAGCCGC |
| A3 | TGAAGC | B2 | CAAGAAC |
| A4 | ACTTGC | B3 | AGTTGGC |
| A5 | TCACAC | B4 | TATCAAC |
| A6 | CGTGAC | B5 | AGGCGGC |
| A7 | ACGCGC | B6 | CGGTATC |
| A8 | CCTCTC | B7 | TGACGAC |
| A9 | ACTCAC | B8 | ACAAGGC |
| A10 | AGACAC | B9 | AGACCTC |
| A11 | CGACTC | B10 | ATACCAC |

### Sequencing Techniques

As described herein, the present methods can be used in conjunction with a variety of sequencing techniques. In some embodiments, the process to determine the nucleotide sequence of a target nucleic acid can be an automated process.

Templates (e.g., nucleic acids and fragments thereof) may be amplified on beads, for example using emulsion PCR methods. In order to use emulsion based amplification techniques with a single template per emulsion bubble, a single primer is attached to the bead, and a single primer is in solution, thereby amplifying the templates such that one end of the duplex is attached to the bead. The hybridized strand can be removed by denaturing the duplex, thereby leaving the immobilized single strand on the bead. The single stranded templates can be captured onto a surface via primers complementary to the templates. Exemplary emulsion-based amplification techniques that can be used in a method of the invention are described in US 2005/0042648; US 2005/0079510; US 2005/0130173 and WO 05/010145, each of which is incorporated herein by reference in its entirety and for all purposes.

Templates can be amplified on a surface using bridge amplification to form nucleic acid clusters. Bridge amplification gives a double stranded template where both ends are immobilized. Methods of generating nucleic acid clusters for use in high-throughput nucleic acid technologies have been described, as noted above. See, for example, U.S. Pat. No. 7,115,400, U.S. Patent Application Publication Nos. 2005/0100900 and 2005/0059048, and PCT Publication Nos. WO 98/44151, WO 00/18957, WO 02/46456, WO 06/064199, and WO 07/010,251, each of which is incorporated by reference herein in its entirety and for all purposes.

Some embodiments include sequencing by synthesis (SBS) techniques. SBS techniques generally involve the enzymatic extension of a nascent nucleic acid strand through the iterative addition of nucleotides or oligonucleotides against a template strand. In traditional methods of SBS, a single nucleotide monomer may be provided to a target nucleotide in the presence of a polymerase in each delivery.

SBS can utilize nucleotide monomers that have a terminator moiety or those that lack any terminator moieties. Methods utilizing nucleotide monomers lacking terminators include, for example, pyrosequencing and sequencing using .gamma.-phosphate-labeled nucleotides. In methods using nucleotide monomers lacking terminators, the number of different nucleotides added in each cycle can be dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.). In preferred methods a terminator moiety can be reversibly terminating.

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other. For example, the different nucleotides present in a sequencing reagent can have different labels and they can be distinguished using appropriate optics as exemplified by the sequencing methods developed by Solexa (now Illumina, Inc.).

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1):84-9; Ronaghi, M. (2001) “Pyrosequencing sheds light on DNA sequencing.” Genome Res. 11(1):3-11; Ronaghi, M., Uhlen, M. and Nyren, P. (1998) “A sequencing method based on real-time pyrophosphate.” Science 281(5375):363; U.S. Pat. No. 6,210,891; U.S. Pat. No. 6,258,568 and U.S. Pat. No. 6,274,320, the disclosures of which are incorporated herein by reference in their entireties and for all purposes). In pyrosequencing, released PPi can be detected by being immediately converted to adenosine triphosphate (ATP) by ATP sulfurylase, and the level of ATP generated is detected via luciferase-produced photons.

In another example type of SBS, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,163 and U.S. Pat. No. 7,057,026, the disclosures of which are incorporated herein by reference and for all purposes. This approach is being commercialized by Solexa (now Illumina Inc.), and is also described in WO 91/06678 and WO 07/123,744 (filed in the United States patent and trademark Office as U.S. Ser. No. 12/295,337), each of which is incorporated herein by reference in their entireties and for all purposes. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199 and PCT Publication No. WO 07/010,251, the disclosures of which are incorporated herein by reference in their entireties and for all purposes.

Some embodiments can utilize sequencing by ligation techniques. Such techniques utilize DNA ligase to incorporate nucleotides and identify the incorporation of such nucleotides. Example ligation-based systems and methods which can be utilized with the methods and systems described herein are described in U.S. Pat. No. 6,969,488, U.S. Pat. No. 6,172,218, and U.S. Pat. No. 6,306,597, the disclosures of which are incorporated herein by reference in their entireties and for all purposes.

Some embodiments can utilize nanopore sequencing (Deamer, D. W. & Akeson, M. “Nanopores and nucleic acids: prospects for ultrarapid sequencing.” Trends Biotechnol. 18:147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties and for all purposes). In such embodiments, the target nucleic acid or nucleotides released from the target nucleic acid pass through a nanopore. The nanopore can be a synthetic pore or biological membrane protein, such as .alpha.-hemolysin. As the target nucleic acid or nucleotides pass through the nanopore, each base-pair (or base) can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53:1996-2001 (2007); Healy, K. “Nanopore-based single-molecule DNA analysis.” Nanomed. 2:459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130:818-820 (2008), the disclosures of which are incorporated herein by reference in their entireties and for all purposes).

Some embodiments can utilize methods involving the real-time monitoring of DNA polymerase activity. Nucleotide incorporations can be detected through fluorescence resonance energy transfer (FRET) interactions between a fluorophore-bearing polymerase and .gamma.-phosphate-labeled nucleotides as described, for example, in U.S. Pat. No. 7,329,492 and U.S. Pat. No. 7,211,414 (each of which is incorporated herein by reference in their entireties and for all purposes) or nucleotide incorporations can be detected with zero-mode waveguides as described, for example, in U.S. Pat. No. 7,315,019 (which is incorporated herein by reference in its entirety and for all purposes) and using fluorescent nucleotide analogs and engineered polymerases as described, for example, in U.S. Pat. No. 7,405,281 and U.S. Patent Application Publication No. 2008/0108082 (each of which is incorporated herein by reference in their entireties and for all purposes). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299:682-686 (2003); Lundquist, P. M. et al. “Parallel confocal detection of single molecules in real time.” Opt. Lett. 33:1026-1028 (2008); Korlach, J. et al. “Selective aluminum passivation for targeted immobilization of single DNA polymerase molecules in zero-mode waveguide nanostructures.” Proc. Natl. Acad. Sci. USA 105:1176-1181 (2008), the disclosures of which are incorporated herein by reference in their entireties and for all purposes). In one example single molecule, real-time (SMRT) DNA sequencing technology provided by Pacific Biosciences Inc. can be utilized with the methods described herein. In some embodiments, a SMRT chip or the like may be utilized (U.S. Pat. Nos. 7,181,122, 7,302,146, 7,313,308, incorporated by reference in their entireties and for all purposes). A SMRT chip comprises a plurality of zero-mode waveguides (ZMW). Each ZMW comprises a cylindrical hole tens of nanometers in diameter perforating a thin metal film supported by a transparent substrate. When the ZMW is illuminated through the transparent substrate, attenuated light may penetrate the lower 20-30 nm of each ZMW creating a detection volume of about 1.times.10-21 L. Smaller detection volumes increase the sensitivity of detecting fluorescent signals by reducing the amount of background that can be observed.

An additional example of a sequencing platform that may be used in association with some of the embodiments described herein is provided by Helicos Biosciences Corp. In some embodiments, TRUE SINGLE MOLECULE SEQUENCING (tSMS)™. can be utilized (Harris T. D. et al., “Single Molecule DNA Sequencing of a viral Genome” Science 320:106-109 (2008), incorporated by reference in its entirety and for all purposes). In one embodiment, a library of target nucleic acids can be prepared by the addition of a 3′ poly(A) tail to each target nucleic acid. The poly(A) tail hybridizes to poly(T) oligonucleotides anchored on a glass cover slip. The poly(T) oligonucleotide can be used as a primer for the extension of a polynucleotide complementary to the target nucleic acid. In one embodiment, fluorescently-labeled nucleotide monomer, namely, A, C, G, or T, are delivered one at a time to the target nucleic acid in the presence DNA polymerase. Incorporation of a labeled nucleotide into the polynucleotide complementary to the target nucleic acid is detected, and the position of the fluorescent signal on the glass cover slip indicates the molecule that has been extended. The fluorescent label is removed before the next nucleotide is added to continue the sequencing cycle. Tracking nucleotide incorporation in each polynucleotide strand can provide sequence information for each individual target nucleic acid.

An additional example of a sequencing platform that can be used in association with the methods described herein is provided by Complete Genomics Inc. Libraries of target nucleic acids can be prepared where target nucleic acid sequences are interspersed approximately every 20 by with adaptor sequences. The target nucleic acids can be amplified using rolling circle replication, and the amplified target nucleic acids can be used to prepare an array of target nucleic acids. Methods of sequencing such arrays include sequencing by ligation, in particular, sequencing by combinatorial probe-anchor ligation (cPAL).

In some embodiments using cPAL, about 10 contiguous bases adjacent to an adaptor may be determined. A pool of probes that includes four distinct labels for each base (A, C, T, G) is used to read the positions adjacent to each adaptor. A separate pool is used to read each position. A pool of probes and an anchor specific to a particular adaptor is delivered to the target nucleic acid in the presence of ligase. The anchor hybridizes to the adaptor, and a probe hybridizes to the target nucleic acid adjacent to the adaptor. The anchor and probe are ligated to one another. The hybridization is detected and the anchor-probe complex is removed. A different anchor and pool of probes is delivered to the target nucleic acid in the presence of ligase.

The sequencing methods described herein can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail herein.

Methods for amplification of nucleic acids are well known in the art. Any appropriate method of amplification may be used in conjunction with the methods disclosed herein. For example, a useful amplification technique is PCR (polymerase chain reaction). Methods of PCR include basic PCR (Saiki et al., Science 1985, 230:1350-1354), real-time PCR (RT-PCR) (Nanashima et al., J. Biol. Chem. 2008, 283:16868-16875), hot-start PCR (Carothers et al., Biotechniques 1989, 7:494-9 1989; Krishnan et al. Nucl. Acids Res. 1991, 19:1153; Clark, Nucl. Acids Res. 1988, 16:9677-86; Lin & Jayasena, J. Mol. Biol. 1997, 271:100-11; Dang & Jayasena, J. Mol. Biol. 1996, 264:268-78; Scalice et al. J. Immunol. Methods, 1994, 172:147-63; Sharkey et al., Biotechnology 1994, 12:506-9; Moretti, T. et al., BioTechniques 1998, 25:716-22), long PCR (Barnes, Proc. Natl. Acad. Sci. USA 1994, 91:2216-20), quantitative endpoint PCR (Gaudette & Crain, Nucl. Acids Res. 1991, 19:1879-84; Murphy et al., Biochemistry 1990, 29:10351-10356), quantitative real-time PCR (Lee et al., Nucl. Acids Res. 1993, 21:3761-3766; Bernard et al., Anal. Biochem. 1998, 255:101-107; Sherrill et al., J. Am. Chem. Soc. 2004, 126:4550-4556; Frackman et al., Promega Notes 2006, 92:10-13); rapid amplified polymorphic DNA analysis (McClelland & Welsh, PCR Methods Appl. 1994, 4:S59-65; Power, J. Hosp. Infect. 1996, 34:247-265; Black, 1993), rapid amplification of cDNA ends (Troutt et al., Proc. Natl. Acad. Sci. USA 1992, 89:9823-9825; Edwards et al., Methods in Molecular Biology (Vol. 15), White, B. A., ed., Humana Press, Totowa, N.J., 1991; Liu & Gorovsky, Nucl. Acids Res. 1993, 21:4954-60; Fromont-Racine et al., Nucl. Acids Res. 1993, 21:1683-1684), differential display PCR (Liang & Pardee, Science 1992, 257:967-71), in situ PCR (Haase et al., Proc. Natl. Acad. Sci. USA 1990, 87:4971-4975), and high fidelity PCR (Cline et al., Nucl. Acids Res. 1996, 24:3546-3551).

Other means of amplifying nucleic acid that can be used in the methods of the provided invention include, for example, reverse transcription-PCR, real-time PCR, quantitative real-time PCR, digital PCR (dPCR), digital emulsion PCR (dePCR), clonal PCR, amplified fragment length polymorphism PCR (AFLP PCR), allele specific PCR, assembly PCR, asymmetric PCR (in which a great excess of primers for a chosen strand is used), colony PCR, helicase-dependent amplification (HDA), Hot Start PCR, inverse PCR (IPCR), in situ PCR long PCR (extension of DNA greater than about 5 kilobases), multiplex PCR, nested PCR (uses more than one pair of primers), single-cell PCR, touchdown PCR, loop-mediated isothermal PCR (LAMP), and nucleic acid sequence based amplification (NASBA). Other amplification schemes include: Ligase Chain Reaction, Branch DNA Amplification, Rolling Circle Amplification. Circle to Circle Amplification, SPIA amplification, Target Amplification by Capture and Ligation (TACL) amplification, and RACE amplification.

Nucleic acid molecules can be amplified on beads, for example using emulsion PCR methods. Exemplary emulsion-based amplification techniques that can be used in a method disclosed herein are described in US 2005/0042648; US 2005/0079510; US 2005/0130173 and WO 05/010145, each of which is incorporated herein by reference in its entirety and for all purposes. As further described herein, nucleic acid molecules can be amplified on a surface using bridge amplification to form nucleic acid clusters. Exemplary methods of generating nucleic acid clusters for use in high-throughput nucleic acid technologies have been described. See, for example, U.S. Pat. No. 7,115,400, U.S. Patent Application Publication Nos. 2005/0100900 and 2005/0059048, and PCT Publication Nos. WO 98/44151, WO 00/18957, WO 02/46456, WO 06/064199, and WO 07/010,251, each of which is incorporated by reference herein in its entirety and for all purposes.

RNA EXTRACTION. The RNA may be obtained from a cell using techniques known in the art. Typically, the cell is lysed and the RNA is recovered using known nucleic acid purification techniques. Thus, a method set forth herein includes lysing the T cell, thereby providing the plurality of nucleic acids (e.g., RNA molecules).

### Tables

TABLE 1
Patient and treatment characteristics

| Stem | Time | Patient | | Condi- | | Acute | | | Active |
|---|---|---|---|---|---|---|---|---|---|
| cell | Post- | #, Sex, | | tion- | | GVHD | Chronic | Active | systemic |
| source^{a} | HSCT | Age | Disease ^{b} | ing^{c} | Donor^{d} | (grade) | GVHD | GVHD | therapy^{e} |
| Conv | 6 mo | 1, M, 46 | FL | Rtx/Cy/ | MRD | — | — | No | — |
| | | | | Flu/TBI | | | | | |
| | 6 mo | 2, M, 53 | CLL/ | Rtx/Cy/ | MMUD | — | — | No | Tacro |
| | | | SLL | Flu/TBI | | | | | |
| | 12 mo | 3, F, 53 | HL | Flu/Mel | MUD | 2 | — | No | MMF/Siro |
| | 12 mo | 4, M, 70 | MDS | Cy/Flu/ | MRD | 1 | lim | Yes | Tacro |
| | | | | Thio/TBI | | | | | |
| | 12 mo | 5, M, 51 | NHL | Cy/Thio/ | MRD | 2 | ext | No | Tacro |
| | | | | TBI | | | | | |
| | 12 mo | 6, F, 23 | ALL | Cy/Thio/ | MUD | 2 | — | No | Tacro |
| | | | | TBI | | | | | |
| | 12 mo | 7, M, 40 | AML | Clo/Mel/ | MUD | 2 | limⁱ | Yes | Tacro |
| | | | | Thio | | | | | |
| TCD | 12 mo | 1, F, 39 | CML | Cy/Thio/ | MUD | — | — | No | — |
| | | | | TBI | | | | | |
| | 6 mo | 2, M, 57 | AML | Clo/Mel/ | MMUD ^{h} | — | — | No | — |
| | | | | Thio | | | | | |
| | 6 mo | 3, F, 41 | MDS | Bu/Flu/ | MUD | — | — | No | — |
| | | | | Mel | | | | | |
| | 6 mo | 4, F, 63 | AML | Bu/Flu/ | MRD | — | — | No | — |
| | | | | Mel | | | | | |
| | 6 mo | 5, F, 65 | MDS | Bu/Flu/ | MMUD | — | — | No | — |
| | | | | Mel | | | | | |
| | 6 mo | 6, F, 56 | MDS | Bu/Flu/ | MMUD | — | — | No | — |
| | | | | Mel | | | | | |
| | 12 mo | 7, M, 36 | AML | Cy/Thio/ | MMUD | — | — | No | — |
| | | | | TBI | | | | | |
| | 12 mo | 8, M, 67 | AML | Bu/Flu/ | MRD | — | — | No | — |
| | | | | Mel | | | | | |
| | 12 mo | 9, M, 56 | CML | Cy/Thio/ | MUD | 2 | — | No | — |
| | | | | TBI | | | | | |
| | 12 mo | 10, M, 42 | MDS | Bu/Flu/ | MRD | 1 | lim^{j} | Yes | — |
| | | | | Mel | | | | | |
| | 12 mo | 11, F, 48 | MM | Bu/Flu/ | MUD | — | — | No | — |
| | | | | Mel | | | | | |
| DUCB | 6 mo | 1, F, 59 | AML | Cy/Flu/ | DUCB | 1 | — | No | CSA/ |
| | | | | Thio/TBI | | | | | MMF |
| | 6 mo | 2, F, 24 | ALL | Cy/Flu/ | DUCB | 2 | — | No | CSA/ |
| | | | | TBI | | | | | MMF |
| | 6 mo | 3, M, 36 | HL | Cy/Flu/ | DUCB | 2 | — | No | MMF/ |
| | | | | TBI | | | | | Siro |
| | 6 mo | 4, F, 44 | AML | Cy/Flu/ | DUCB | 2 | -- | No | CSA/ |
| | | | | Thio/TBI | | | | | MMF |
| | 6 mo | 5, M, 54 | AML | Cy/Flu/ | DUCB | 3 | — | No | CSA/ |
| | | | | Thio/TBI | | | | | MMF |
| | 12 mo | 6, M, 59 | MDS | Cy/Flu/ | DUCB | 2 | — | No | CSA/ |
| | | | | Thio/TBI | | | | | MMF |
| DUCB | 12 mo | 7, M, 34 | ALL | Cy/Flu/ | DUCB | 3 | — | No | CSA/ |
| | | | | TBI | | | | | MMF |
| | 12 mo | 8, F, 25 | ALL | Cy/Flu/ | DUCB | 3 | — | No | CSA/ |
| | | | | TBI | | | | | MMF |
| | 12 mo | 9, M, 51 | MZL | Cy/Flu/ | DUCB | 2 | ext | No | MMF |
| | | | | TBI | | | | | |
| | 12 mo | 10, M, 49 | DLBCL | Cy/Flu/ | DUCB | 2 | — | Yes ^{k} | CSA/ |
| | | | | Thio/TBI | | | | | MMF |
| Healthy | — | 1, M, 56 | n/a | | | | | | |
| | — | 2, M, 30 | n/a | | | | | | |
| | — | 3, F, 38 | n/a | | | | | | |
| | — | 4, M, 44 | n/a | | | | | | |
| | — | 5, M, 61 | n/a | | | | | | |

| | | | | | | | Post- |
|---|---|---|---|---|---|---|---|
| | | | | | CD4 | | trans- |
| Stem | Time | Patient | Prior | | CD4 | | plant |
| cell | Post- | #, Sex, | systemic | CD4 | 5RA | CD8 | in- |
| source^{a} | HSCT | Age | steroids | count^{f} | count^{f} | count^{f} | fection^{g} |
| Conv | 6 mo | 1, M, 46 | No | 828 | 110 | 3,918 | — |
| | 6 mo | 2, M, 53 | Yes^{l} | 418 | 0 | 1,627 | BK, |
| | | | | | | | HSV, |
| | | | | | | | RV |
| | 12 mo | 3, F, 53 | Yes | 233 | 35 | 146 | — |
| | 12 mo | 4, M, 70 | No | 592 | 56 | 324 | — |
| | 12 mo | 5, M, 51 | No | 701 | 220 | 400 | CMV^{m} |
| | 12 mo | 6, F, 23 | Yes^{l} | 452 | 71 | 1,810 | CMV^{m}, |
| | | | | | | | BK |
| | 12 mo | 7, M, 40 | Yes | 328 | 82 | 396 | — |
| TCD | 12 mo | 1, F, 39 | No | 116 | 0 | 101 | CMV^{m}, |
| | | | | | | | EBVⁿ |
| | 6 mo | 2, M, 57 | No | 90 | 16 | 98 | — |
| | 6 mo | 3, F, 41 | No | 431 | 15 | 816 | CMV^{m} |
| | 6 mo | 4, F, 63 | No | 163 | 0 | 1,714 | BK, |
| | | | | | | | CMV^{m} |
| | 6 mo | 5, F, 65 | No | 258 | 0 | 354 | CMV^{m} |
| | 6 mo | 6, F, 56 | No | 51 | 0 | 51 | CMV^{m} |
| | 12 mo | 7, M, 36 | No | 195 | 41 | 164 | RSV |
| | 12 mo | 8, M, 67 | No | 212 | 6 | 25 | EBVⁿ |
| | 12 mo | 9, M, 56 | No | 112 | 9 | 215 | — |
| | 12 mo | 10, M, 42 | No | 464 | 69 | 584 | — |
| | 12 mo | 11, F, 48 | No | 154 | 19 | 317 | CMV^{m}, |
| | | | | | | | FLU |
| DUCB | 6 mo | 1, F, 59 | No | 280 | 20 | 860 | CMV^{m}, |
| | | | | | | | HHV6 |
| | 6 mo | 2, F, 24 | Yes^{l} | 168 | 18 | 42 | CMV^{m}, |
| | | | | | | | HHV6, |
| | | | | | | | RSV |
| | 6 mo | 3, M, 36 | Yes^{l} | 404 | 135 | 174 | HHV6, |
| | | | | | | | RV |
| | 6 mo | 4, F, 44 | No | 246 | 19 | 9 | — |
| | 6 mo | 5, M, 54 | Yes^{l} | 273 | 59 | 46 | BK, |
| | | | | | | | HHV6, |
| | 12 mo | 6, M, 59 | No | 486 | 47 | 19 | EBV^{o} |
| DUCB | 12 mo | 7, M, 34 | Yes | 129 | 86 | 387 | CMV^{m,p}, |
| | | | | | | | BK |
| | 12 mo | 8, F, 25 | Yes | 279 | 56 | 68 | — |
| | 12 mo | 9, M, 51 | Yes | 223 | 223 | 10 | MPV^{p} |
| | 12 mo | 10, M, 49 | No | 346 | 45 | 32 | BK |
| Healthy | — | 1, M, 56 | | | | | |
| | — | 2, M, 30 | | | | | |
| | — | 3, F, 38 | | | | | |
| | — | 4, M, 44 | | | | | |
| | — | 5, M, 61 | | | | | |

^{a}Conv, Conventional peripheral blood stem cell graft; TCD, T-cell-depleted peripheral blood stem cell graft; DUCB, Double-unit umbilical cord blood graft; Healthy, Healthy donor.
^{b} NHL, Non-Hodgkin's lymphoma; FL, follicular lymphoma; MZL, Marginal zone lymphoma; SLL, Small lymphocytic lymphoma; DLBCL, Diffuse large B-cell lymphoma; CLL, chronic lymphocytic leukemia; HL,
Hodgkin lymphoma; MDS, Myelodysplastic syndrome; ALL, Acute lymphoblastic leukemia; AML, Acute myeloid leukemia; CML, Chronic myeloid leukemia; MM, Multiple myeloma.
^{c}Cy, Cyclophosphamide; Flu, Fludarabine; TBI, Total body irradiation; Rtx, Rituximab; Mel, Melphalan; Thio, Thiotepa; Clo, Clofarabine; Bu, Busulfan.
^{d}MRD, Matched related donor; MMUD, mismatched unrelated donor; MUD, Matched unrelated donor.
^{e}Tacro, Tacrolimus; MMF, Mycophenolate mofetil; Siro, Sirolimus; CSA, Cyclosporine-A.
^{f}Cells/1.
^{g}BK, BK polyomavirus; HSV, Herpes simplex virus; RV, Rhinovirus; CMV, Cytomegalovirus; EBV, Epstein-Barr virus; RSV, Respiratory syncytial virus; FLU, Influenza virus; HHV6, Human Herpesvirus 6; RV, Rotavirus; MPV, Metapneumovirus.
^{h} T-cell-depleted bone marrow graft.
ⁱMild oral and eye symptoms only.
^{j}Mild eye symptoms only.
^{k} Ongoing acute GVHD.
^{l}Active steroids.
^{m}All patients with CMV reactivation were successfully treated for CMV viremia and did not progress to CMV disease.
ⁿPatients had documented EBV viremia and post-transplant lymphoproliferative disease; and were treated with rituximab.
^{o}Patient had documented EBV viremia, but no documented post-transplant lymphoproliferative disease; and was treated with rituximab.
^{p}Infection within three months of transplantation. n/a, not applicable; lim, limited; ext, extensive.

TABLE 2
UPDATED DATA (March 2013)

| | Last | | | | | | | Active |
|---|---|---|---|---|---|---|---|---|
| Stem cell | Follow | Patient #, | Acute GVHD | | Chronic | Active GVHD at | Prior systemic | systemic |
| source^{a} | up | Sex, Age | (Type/Type) | onset | GVHD | last follow up | steroids | therapy^{e} |
| Conv | Jan. 24, 2013 | 1, M, 46 | 0 | | Mild (mouth) | No | — | — |
| | Feb. 21, 2013 | 2, M, 53 | Late onset-2 | Jun. 7, 2012 | No | | Yes | Tacro |
| | Apr. 7, 2012 | 3, F, 53 | Late onset-2 | Nov. 16, 2010 | No | No | Yes for BOOP | Siro PDN (20 |
| | | | | | | | | mg/d), |
| | | | | | | | | Budesonide |
| | Feb. 18, 2013 | 4, M, 70 | Late onset-2 | May 21, 2011 | Moderate | Yes | Yes | PDN, Tacro, |
| | | | | | | skin/musculoskelet | | MMF |
| | | | | | | al sclerotic | | |
| | | | | | | cGVHD | | |
| | Jan. 24, 2013 | 5, M, 51 | Late onset-2 | Nov. 11, 2010 | 0 | Yes, | No | Siro, MMF, |
| | | | | | | hyperpigmentation | | eye drops, |
| | | | | | | skin, mouth | | Cyclosporine |
| | | | | | | hypersensitivity, | | drops |
| | | | | | | dry eyes | | |
| | Feb. 01, 2013 | 6, F, 23 | 0 | — | 0 | No | No | — |
| | Feb. 28, 2013 | 7, M, 40 | 0 | — | Mild (mouth) | Yes | No | Tacro, |
| | | | | | | | | cyclosporine |
| | | | | | | | | oral rinse |
| TCD | Feb. 26, 2013 | 1, F, 39 | 0 | — | Moderate after | Yes | No | — |
| | | | | | DLI^{a} | hyperpigmentation | | |
| | | | | | | skin, mouth | | |
| | | | | | | hypersensitivity | | |
| | Oct. 24, 2012 | 2, M, 57 | 0 | — | 0 | No | No | — |
| | Mar. 5, 2013 | 4, F, 63 | 0 | — | 0 | No | No | — |
| | Nov. 20, 2012 | 5, F, 65 | 0 | — | 0 | No | No | — |
| | Nov. 09, 2012 | 6, F, 56 | Late onset-2 | Mar. 5, 2012 | 0 | No | No | — |
| | Sep. 26, 2012 | 7, M, 36 | 0 | — | 0 | No | No | — |
| | Oct. 12, 2012 | 8, M, 67 | 0 | — | 0 | No | No | — |
| | Dec. 14, 2012 | 9, M, 56 | 0 | — | 0 | No | No | — |
| | Dec. 5, 2011 | 10, M, 42 | 0 | — | Mild ^{b} | Yes, dry eyes | No | — |
| | Dec. 12, 2012 | 11, F, 48 | 0 | — | 0 | No | No | — |
| DUCB | Mar. 1, 2013 | 1, F, 59 | 0 | — | 0 | No | No | — |
| | Feb. 22, 2013 | 2, F, 24 | 2 | Sep. 28, 2011 | 0 | Yes | No | PDN |
| DUCB | Nov. 27, 2012 | 3, M, 36 | 0 | — | 0 | No | No | — |
| | Jan. 16, 2013 | 4, F, 44 | 0 | — | 0 | No | No | — |
| | Mar. 5, 2013 | 5, M, 54 | Late onset-3 | Nov. 26, 2012 | 0 | Yes | No | CSA/MMF, |
| | | | | | | | | PDN |
| | Jan. 9, 2013 | 6, M, 59 | Late onset-2 | Sep. 17, 2010 | 0 | No | No | CSA/MMF |
| | Jan. 9, 2013 | 7, M, 34 | 0 | — | 0 | No | No | CSA/Budesonide |
| | Jan. 11, 2013 | 8, F, 25 | Late onset-2 | Apr. 4, 2012 | 0 | Yes | No | — |
| | 12 mo | 9, M, 51 | | | | | | MMF |
| | 12 mo | 10, M, 49 | | | | | | CSA/MMF |

^{a}On protocol 07-127 (IL-7 9/21-28 Oct. 15, 2010). Bcr/abl pcr+ Jan. 25, 11. DLI Sep. 28, 2011. Moderate cGVHD after DLL
^{b} Dry eyes

TABLE 3
HLA ANTIGENS

| Stem cell | Patient #, | | | | | | |
|---|---|---|---|---|---|---|---|
| source^{a} | Sex, Age | Patient HLA | | | Donor HLA | | |
| Conv | 1, M, 46 | A | BMMP | 02 01 | A | BMMP | 02 01 |
| | | B | 52 01 | 44 03 | B | 52 01 | 44 03 |
| | | Cw | 12 02 | 03 04 | Cw | 12 02 | 03 04 |
| | | DRB1 | 15 02 | 07 01 | DRB1 | 15 02 | 07 01 |
| | | DQ1 | 06 01 | 02 02 | DQ1 | 06 01 | 02 02 |
| | 2, M, 53 | A | 0101/0101 | 24 02 | A | 0101/0101N | 24 02 |
| | | B | N | 57 01 | B | 38 01 | 57 01 |
| | | Cw | 38 01 | 06 02 | Cw | 06 02 | 12 03 |
| | | DRB1 | 06 02 | 07 01 | DRB1 | 01 01 | 07 01 |
| | | DQ1 | 01 01 | 03 03 | DQ1 | 05 01 | 03 03 |
| | | | 05 01 | | | | |
| | 3, F, 53 | A | 0301/0301 | 26 01 | A | 0301/0301N | 26 01 |
| | | B | N | 55 01 | B | 0702/0761 | 55 01 |
| | | Cw | 0702/0761 | 0303/0320N | Cw | 0702/0750 | 0303/0320N |
| | | DRB1 | 0702/0750 | 16 01 | DRB1 | 15 01 | 16 01 |
| | | DQB1 | 15 01 | 05 02 | DQB1 | 06 02 | 05 02 |
| | | | 06 02 | | | | |
| | 4, M, 70 | A | 0301/0301 | 24 02 | A | 0301/0301N 13 | 24 02 |
| | | B | N 13 02 | 40 02 | B | 02 | 40 02 |
| | | Cw | 06 02 | 02 02 | Cw | 06 02 | 02 02 |
| | | DRB1 | 07 01 | 11 01 | DRB1 | 07 01 | 11 01 |
| | | DQB1 | 02 02 | 03 01 | DQB1 | 02 02 | 03 01 |
| Conv | 5, M, 51 | A | 11 01 | 24 02 | A | 11 01 | 24 02 |
| | | B | 4402/4419 | 52 01 | B | 4402/4419N | 52 01 |
| | | Cw | N | 16 04 | Cw | 12 02 | 16 04 |
| | | DRB1 | 12 02 | 15 02 | DRB1 | 11 04 | 15 02 |
| | | DQB1 | 11 04 | 06 01 | DQB1 | 03 01 | 06 01 |
| | | | 03 01 | | | | |
| | 6, F, 23 | A | 02 01 | 2301/2317 | A | 02 01 | 2301/2317 |
| | | B | 0702/0761 | 50 01 | B | 0702/0761 | 50 01 |
| | | Cw | 0702/0750 | 06 02 | Cw | 0702/0750 | 06 02 |
| | | DRB1 | 03 01 | 15 01 | DRB1 | 03 01 | 15 01 |
| | | DQB1 | 02 01 | 06 02 | DQB1 | 02 01 | 06 02 |
| | 7, M, 40 | A | 26 01 | 02 04 | A | 26 01 | 02 04 |
| | | B | 08 01 | 51 01 | B | 08 01 | 51 01 |
| | | Cw | 0701/06/18 | 15 02 | Cw | 0701/06/ | 15 02 |
| | | DRB1 | 15 01 | 04 11 | DRB1 | 18 | 04 11 |
| | | DQB1 | 06 02 | 04 02 | DQB1 | 15 01 | 04 02 |
| | | | | | | 06 02 | |
| TCD | 1, F, 39 | A | 02 01 | 02 01 | A | 02 01 | 02 01 |
| | | B | 0702/0761 | 1501/1501N | B | 0702/07 | 1501/1501N |
| | | Cw | 0702/0750 | 0303/0320N | Cw | 61 | 0303/0320N |
| | | DRB1 | 15 01 | 15 01 | DRB1 | 0702/07 | 15 01 |
| | | DQB1 | 06 02 | 06 02 | DQB1 | 50 | 06 02 |
| | | | | | | 15 01 | |
| | | | | | | 06 02 | |
| | 2, M, 57 | A | 68 FKZ | 25 01 | A | 68 FKZ | 01 BMMP |
| | | B | 57 01 | 40 02 | B | 57 01 | 40 02 |
| | | Cw | 06 02 | 02 02 | Cw | 06 02 | 02 02 |
| | | DRB1 | 07 01 | 04 04 | DRB1 | 07 01 | 04 04 |
| | | DQB1 | 03 03 | 03 02 | DQB1 | 03 03 | 03 02 |
| | 3, F, 41 | A | 68 FKZ | 25 01 | A | 68 FKZ | 25 01 |
| | | B | 57 01 | 40 02 | B | 57 01 | 40 02 |
| | | Cw | 06 02 | 02 02 | Cw | 06 02 | 02 02 |
| | | DRB1 | 07 01 | 04 04 | DRB1 | 07 01 | 04 04 |
| | | DQB1 | 03 03 | 03 02 | DQB1 | 03 03 | 03 02 |
| | 4, F, 63 | A | 02 07 | 24 07 | A | 02 07 | 24 07 |
| | | B | 51 01 | 35 05 | B | 51 01 | 35 05 |
| | | Cw | 15 02 | 04 CXBM | Cw | 15 02 | 04 CXBM |
| | | DRB1 | 14 05 | 15 02 | DRB1 | 14 05 | 15 02 |
| | | DQB1 | 05 03 | 05 02 | DQB1 | 05 03 | 05 02 |
| | 5, F, 65 | A | 24 02 | 24 02 | A | 24 02 | 24 02 |
| | | B | 14 02 | 27 07 | B | 14 02 | 27 07 |
| | | Cw | 02 02 | 15 02 | Cw | 08 02 | 15 02 |
| | | DRB1 | 01 02 | 11 04 | DRB1 | 01 02 | 11 04 |
| | | DQB1 | 05 01 | 03 01 | DQB1 | 05 01 | 03 01 |
| | 6, F, 56 | A | 01 | 31 01 | A | 01 BMMP | 31 01 |
| | | B | BMMP | 57 03 | B | 08 01 | 57 03 |
| | | Cw | 08 01 | 07 WTR | Cw | 07 WTR | 07 WTR |
| | | DRB1 | 07 WTR | 11 03 | DRB1 | 03 01 | 11 01 |
| | | DQB1 | 03 01 | 03 01 | DQB1 | 02 01 | 03 01 |
| | | | 02 01 | | | | |
| TCD | 7, M, 36 | A | 11 01 | 30 02 | A | 11 01 | 30 02 |
| | | B | 1801/18 | 40 01 | B | 1801/1817 | 40 01 |
| | | Cw | 17N | 03 04 | Cw | N | 03 04 |
| | | DRB1 | 05 01 | 13 02 | DRB1 | 05 01 | 13 02 |
| | | DQB1 | 03 01 | 06 04 | DQB1 | 03 01 | 06 04 |
| | | | 05 01 | | | 02 01 | |
| | 8, M, 67 | A | 24 02 | 24 02 | A | 24 02 | 24 02 |
| | | B | 1501/15 | 1501/1501N | B | 1501/1501 | 1501/1501N |
| | | Cw | 01N | 0303/0320N | Cw | N | 0303/0320N |
| | | DRB1 | 01 02 | 13 01 | DRB1 | 01 02 | 13 01 |
| | | DQB1 | 09 01 | 06 03 | DQB1 | 09 01 | 06 03 |
| | | | 03 03 | | | 03 03 | |
| | 9, M, 56 | A | 02 01 | 2301/2317 | A | 02 01 | 2301/2317 |
| | | B | 1501/15 | 57 01 | B | 1501/1501 | 57 01 |
| | | Cw | 01N | 06 02 | Cw | N | 06 02 |
| | | DRB1 | 03 04 | 07 01 | DRB1 | 03 04 | 07 01 |
| | | DQB1 | 15 01 | 03 03 | DQB1 | 15 01 | 03 03 |
| | | | 06 02 | | | 06 02 | |
| | 10, M, 42 | A | 0301/03 | 33 01 | A | 0301/0301 | 33 01 |
| | | B | 01N | 56 01 | B | N | 56 01 |
| | | Cw | 14 02 | 01 02 | Cw | 14 02 | 01 02 |
| | | DRB1 | 08 02 | 01 02 | DRB1 | 08 02 | 01 02 |
| | | DQB1 | 01 01 | 05 01 | DQB1 | 01 01 | 05 01 |
| | | | 05 01 | | | 05 01 | |
| | 11, F, 48 | A | 0101/01 | 02 01 | A | 0101/0101 | 02 01 |
| | | B | 01N | 0702/0761 | B | N | 0702/0761 |
| | | Cw | 08 01 | 15 02 | Cw | 08 01 | 15 02 |
| | | DRB1 | 0701/06/ | 03 01 | DRB1 | 0701/06/1 | 03 01 |
| | | DQB1 | 18 | 02 01 | DQB1 | 8 | 02 01 |
| | | | 01 01 | | | 01 01 | |
| | | | 05 01 | | | 05 01 | |
| DUCB | 1, F, 59 | A | 03 02 | 68 02 | A | 0301/030 | 68 02 |
| | | B | 51 01 | 53 01 | B | 1N | 5301/01 |
| | | Cw | 15 02 | 04 CXBM | Cw | 5101/01 | 0401/0409N/0430 |
| | | DRB1 | 03 01 | 08 04 | DRB1 | 1402/01 | 1302/01 |
| | | DQB1 | 02 01 | 03 19 | DQB1 | 03 01 | 0604/01 |
| | | | | | | 0201/01 | |
| | 2, F, 24 | A | 31 01 | 30 04 | A | 2402/240 | 3001/01 |
| | | B | 41 02 | 18 RRG | B | 2L | 4101 |
| | | Cw | 17 MN | 07 WTR | Cw | 1302/01 | 1701/02/03 |
| | | DRB1 | 11 04 | 13 03 | DRB1 | 0602 | 13 03 |
| | | DQB1 | 03 01 | 03 01 | DQB1 | 11 04 | 0301/01 |
| | | | | | | 0301/01 | |
| | 3, M, 36 | A | 03 XKS | 23 CJT | A | 02 02 | 03:01/01N |
| | | B | 35 BJTR | 58 02 | B | 3501/42 | 58 02 |
| | | Cw | 04 CXBM | 06 02 | Cw | 04:01/09 | 06 02 |
| | | DRB1 | 01 01 | 11 02 | DRB1 | N/30 | 110201 |
| | | DQB1 | 05 01 | 03 01 | DQB1 | 10101 | 30101 |
| | | | | | | 50101 | |
| | 4, F, 44 | A | 02 01 | 24 02 | A | 02:01/02: | 24 02 00 |
| | | B | 44 PYV | 44 05 | B | 01L | 44 03 00 |
| | | Cw | 05 01 | 02 02 | Cw | 44 02 00 | 0501/0503 |
| | | DRB1 | 07 01 | 11 04 | DRB1 | 0401/040 | 07 01 |
| | | DQB1 | 02 02 | 03 01 | DQB1 | 9N | 03 03 |
| | | | | | | 07 01 | |
| | | | | | | 0201/02/ | |
| | | | | | | 04 | |
| DUCB | 5, M, 54 | A | 02 01 | 68 01 | | | |
| | | B | 35 BJTR | 14 02 | | | |
| | | Cw | 14 02 | 08 02 | | | |
| | | DRB1 | 01 01 | 07 01 | | | |
| | | DQB1 | 05 01 | 03 03 | | | |
| | 6, M, 59 | A | 24 02 | 02 01 | A | 02 01 | 24 02 |
| | | B | 0702/0761 | 51 01 | B | 07 02 | 51 01 |
| | | Cw | 0702/0750 | 15 02 | Cw | 07 02 | 14 02 |
| | | DRB1 | 15 01 | 07 01 | DRB1 | 15 01 | 17 01 |
| | | DQB1 | 06 02 | 02 02 | DQB1 | 02 02 | 06 02 |
| | 7, M, 34 | A | 11 XX | 32 XX | A | 0101/0101 | 1101/01 |
| | | B | 08 01 | 3801/42/57 | B | N | 3501/42 |
| | | Cw | 0702/0750 | 0401/09N/ | Cw | 0801/01 | 0401/0409N/043 |
| | | DRB1 | 03 01 | 30 | DRB1 | 0701/06/1 | 0 |
| | | DQB1 | 02 01 | 13 02 | DQB1 | 8 | 03 01 |
| | | | | 06 09 | | 0101/01 | 0201/01 |
| | | | | | | 0501/01 | |
| | 8, F, 25 | A | 02 01 | 30 01 | A | 0201/0201 | 300201 |
| | | B | 49 01 | 51 01 | B | L | 51 01 |
| | | Cw | 0701/06/18 | 14 02 | Cw | 490101 | 140201 |
| | | DRB1 | 08 01 | 13 05 | DRB1 | 0701/06/1 | 08 01 |
| | | DQB1 | 04 02 | 03 01 | DQB1 | 8 | 04 02 |
| | | | | | | 07 01 | |
| | | | | | | 02 02 | |
| | 9, M, 51 | A | 02 01 | 0301/0301 | A | 02 01 | 0301/0301N |
| | | B | 38 01 | N | B | 38 01 | 44 27 |
| | | Cw | 12 03 | 44 27 | Cw | 12 03 | 0704/0711 |
| | | DRB1 | 13 01 | 0704/0711 | DRB1 | 13 01 | 16 01 |
| | | DQB1 | 06 03 | 15 02 | DQB1 | 06 03 | 05 02 |
| | | | | 06 01 | | | |
| | 10, M, 49 | A | 0101/0101 | 32 01 | A | 0101/01N | 320101 |
| | | B | N | 51 01 | B | 80101 | 510101 |
| | | Cw | 08 01 | 15 02 | Cw | ′0701/06/1 | 10201 |
| | | DRB1 | 0701/06/18 | 12 DUKV | DRB1 | 8 | 1201/06/10/17 |
| | | DQB1 | 03 01 | 03 01 | DQB1 | 03 01 | 30101 |
| | | | 02 01 | | | 20101 | |

Conv-3: Dead on Apr. 7, 2012.
COD: pulmonary failure.
TCD-2: relapse on Feb. 23, 2012.
Dead on Oct. 24, 2012. .
COD: relapse.

### REFERENCES

The contents of each of the following is hereby incorporated by reference as if fully set forth herein, except for terminology that is inconsistent with the terminology used herein.
1. Storek, J., et al. Reconstitution of the immune system after hematopoietic stem cell transplantation in humans. Semin Immunopathol 30, 425-437 (2008).
2. Seggewiss, R. & Einsele, H. Immune reconstitution after allogeneic transplantation and expanding options for immunomodulation: an update. Blood 115, 3861-3868 (2010).
3. Deeg, H. J. & Socie, G. Malignancies after hematopoietic stem cell transplantation: many questions, some answers. Blood 91, 1833-1844 (1998).
4. Small, T. N., et al. Comparison of immune reconstitution after unrelated and related T-cell-depleted bone marrow transplantation: effect of patient age and donor leukocyte infusions. Blood 93, 467-480 (1999).
5. Maury, S., et al. Prolonged immune deficiency following allogeneic stem cell transplantation: risk factors and complications in adult patients. Br J Haematol 115, 630-641 (2001).
6. Nikolich-Zugich, J., Slifka, M. K. & Messaoudi, I. The many important facets of T-cell repertoire diversity. Nat Rev Immunol 4, 123-132 (2004).
7. Goldberg, G. L., Zakrzewski, J. L., Perales, M. A. & van den Brink, M. R. Clinical strategies to enhance T cell reconstitution. Semin Immunol 19, 289-296 (2007).
8. Dumont-Girard, F., et al. Reconstitution of the T-cell compartment after bone marrow transplantation: restoration of the repertoire by thymic emigrants. Blood 92, 4464-4471 (1998).
9. Douek, D. C., et al. Assessment of thymic output in adults after haematopoietic stem-cell transplantation and prediction of T-cell reconstitution. Lancet 355, 1875-1881 (2000).
10. Roux, E., et al. Recovery of immune reactivity after T-cell-depleted bone marrow transplantation depends on thymic activity. Blood 96, 2299-2303 (2000).
11. Weinberg, K., et al. Factors affecting thymic function after allogeneic hematopoietic stem cell transplantation. Blood 97, 1458-1466 (2001).
12. Lynch, H. E., et al. Thymic involution and immune reconstitution. Trends Immunol 30, 366-373 (2009).
13. Langerak, A. W., et al. Molecular and flow cytometric analysis of the Vbeta repertoire for clonality assessment in mature TCRalphabeta T-cell proliferations. Blood 98, 165-173 (2001).
14. Gaspar, H. B., et al. Long-term persistence of a polyclonal T cell repertoire after gene therapy for X-linked severe combined immunodeficiency. Sci Transl Med 3, 97ra79 (2011).
15. Gorski, J., et al. Circulating T cell repertoire complexity in normal individuals and bone marrow recipients analyzed by CDR3 size spectratyping. Correlation with immune status. J Immunol 152, 5109-5119 (1994).
16. Memon, S. A., Sportes, C., Flomerfelt, F. A., Gress, R. E. & Hakim, F. T. Quantitative analysis of T cell receptor diversity in clinical samples of human peripheral blood. J Immunol Methods 375, 84-92 (2012).
17. Verfuerth, S., et al. Longitudinal monitoring of immune reconstitution by CDR3 size spectratyping after T-cell-depleted allogeneic bone marrow transplant and the effect of donor lymphocyte infusions on T-cell repertoire. Blood 95, 3990-3995 (2000).
18. Wu, C. J., et al. Reconstitution of T-cell receptor repertoire diversity following T-cell depleted allogeneic bone marrow transplantation is related to hematopoietic chimerism. Blood 95, 352-359 (2000).
19. Talvensaari, K., et al. A broad T-cell repertoire diversity and an efficient thymic function indicate a favorable long-term immune reconstitution after cord blood stem cell transplantation. Blood 99, 1458-1464 (2002).
20. Klarenbeek, P. L., et al. Human T-cell memory consists mainly of unexpanded clones. Immunol Lett 133, 42-48 (2010).
21. Robins, H. S., et al. Overlap and effective size of the human CD8+ T cell receptor repertoire. Sci Transl Med 2, 47ra64 (2010).
22. Sherwood, A. M., et al. Deep sequencing of the human TCRgamma and TCRbeta repertoires suggests that TCRbeta rearranges after alphabeta and gammadelta T cell commitment. Sci Transl Med 3, 90ra61 (2011).
23. Venturi, V., et al. A mechanism for TCR sharing between T cell subsets and individuals revealed by pyrosequencing. J Immunol 186, 4285-4294 (2011).
24. Warren, R. L., et al. Exhaustive T-cell repertoire sequencing of human peripheral blood samples reveals signatures of antigen selection and a directly measured repertoire size of at least 1 million clonotypes. Genome Res 21, 790-797 (2011).
25. Ponce, D. M., et al. Reduced late mortality risk contributes to similar survival after double-unit cord blood transplantation compared with related and unrelated donor hematopoietic stem cell transplantation. Biol Blood Marrow Transplant 17, 1316-1326 (2011).
26. Jacobson, C. A., et al. Immune reconstitution after double umbilical cord blood stem cell transplantation: comparison with unrelated peripheral blood stem cell transplantation. Biol Blood Marrow Transplant 18, 565-574 (2012).
27. Nguyen, P., et al. Identification of errors introduced during high throughput sequencing of the T cell receptor repertoire. BMC Genomics 12, 106 (2011).
28. Venturi, V., Kedzierska, K., Turner, S. J., Doherty, P. C. & Davenport, M. P. Methods for comparing the diversity of samples of the T cell receptor repertoire. J Immunol Methods 321, 182-195 (2007).
29. Lim, A., et al. Frequent contribution of T cell clonotypes with public TCR features to the chronic response against a dominant EBV-derived epitope: application to direct detection of their molecular imprint on the human peripheral T cell repertoire. J Immunol 165, 2001-2011 (2000).
30. Sauter, C., et al. Serious infection risk and immune recovery after double-unit cord blood transplantation without antithymocyte globulin. Biol Blood Marrow Transplant 17, 1460-1471 (2011).
31. Bacigalupo, A., et al. Defining the intensity of conditioning regimens: working definitions. Biol Blood Marrow Transplant 15, 1628-1633 (2009).
32. Jakubowski, A. A., et al. T cell depleted stem-cell transplantation for adults with hematologic malignancies: sustained engraftment of HLA-matched related donor grafts without the use of antithymocyte globulin. Blood 110, 4552-4559 (2007).
33. Barker, J. N., et al. A “no-wash” albumin-dextran dilution strategy for cord blood unit thaw: high rate of engraftment and a low incidence of serious infusion reactions. Biol Blood Marrow Transplant 15, 1596-1602 (2009).
34. Rowlings, P. A., et al. IBMTR Severity Index for grading acute graft-versus-host disease: retrospective comparison with Glucksberg grade. Br J Haematol 97, 855-864 (1997).
35. Schloss, P. D., et al. Introducing mothur: open-source, platform-independent, community-supported software for describing and comparing microbial communities. Appl Environ Microbiol 75, 7537-7541 (2009).
36. Brochet, X., Lefranc, M. P. & Giudicelli, V. IMGT/V-QUEST: the highly customized and integrated system for IG and TR standardized V-J and V-D-J sequence analysis. Nucleic Acids Res 36, W503-508 (2008).

1. A method for determining T-cell receptor β clonotype diversity and frequency in a subject, comprising
a) obtaining a blood sample from the subject
b) isolating CD4⁺ and CD8⁺ T-lymphocytes, or subsets of CD4⁺ and CD8⁺ T-lymphocytes
c) extracting total RNA from the cells isolated in step b),
d) generating cDNA from the total RNA,
e) amplifying the cDNA,
f) sequencing the amplified cDNA,
g) identifying T-cell receptor β clonotypes in the cDNA sequences and quantifying the diversity of the clonotypes and the clonotype frequency of each clonotype in the sample.

2. The method of claim 1, wherein quantifying the diversity of the clonotypes comprises using the inverse Simpson's diversity index (1/Ds), which sums the frequency of each clonotype.

3. A method for determining a change in T-cell receptor β clonotype diversity and frequency in a subject over time, comprising
a) obtaining a first blood sample from the subject at a first time point and obtaining a second blood sample at a second later time point,
b) isolating CD4⁺ and CD8⁺ T-lymphocytes or subsets of the CD4⁺ and CD8⁺ T-lymphocytes from each sample,
c) extracting total RNA from the cells isolated in step b) for each sample,
d) generating cDNA from the total RNA for each sample,
e) amplifying the cDNA for each sample,
f) sequencing the amplified cDNA for each sample,
g) identifying T-cell receptor β clonotypes in the cDNA sequences and quantifying the diversity of the clonotypes and frequency of each clonotype in each sample, and
h) determining based on at least one of the diversity of the clonotypes in each sample or the frequency of at least one clonotype in each sample, whether there is a statistically significant increase in T-cell receptor β clonotype diversity or frequency at the second time point, or whether T-cell receptor β clonotype diversity or frequency is not statistically significantly changed at the second time point, or whether there is a statistically significant decrease in T-cell receptor β clonotype diversity or frequency at the second time point.

4. The method of claim 3, wherein quantifying the diversity of the clonotypes comprises using the inverse Simpson's diversity index (1/Ds), which sums the frequency of each clonotype.

5. The method of claim 3, wherein a statistically significant increase in T-cell receptor β clonotype diversity at the second time point indicates an increase in immunocompentence of the subject relative to the first time point, and a statistically significant decrease in T-cell receptor β clonotype diversity at the second time point indicates a decrease in immunocompentence of the subject relative to the first time point.

6. The method of claim 3, wherein a statistically significant increase in frequency of a particular T-cell receptor β clonotype frequency at the second time point indicates expansion of the particular clonotype.

7. The method of claim 1, wherein the subject has had an allogeneic hematopoietic stem cell transplant selected from the group consisting of double-unit umbilical cord blood transplant, T-cell-depleted peripheral blood stem cell transplant, and unmanipulated peripheral blood stem cell transplant.

8. The method of claim 1, wherein the subject has a T-cell disorder selected from the group comprising immunodeficiencies, autoimmune diseases, infectious diseases, inflammatory diseases, cancer, and a precancerous condition.

9. The method of claim 3, wherein the subject has a T-cell disorder selected from the group comprising immunodeficiencies, autoimmune diseases, infectious diseases, inflammatory diseases, cancer, and a precancerous condition, is receiving immunotherapy, and a significant increase in T-cell receptor β clonotype diversity indicates that the immunotherapy is effective.

10. The method of claim 3, wherein the subject has a T-cell disorder selected from the group comprising immunodeficiencies, autoimmune diseases, infectious diseases, inflammatory diseases, cancer, and a precancerous condition, is receiving immunotherapy, and a significant decrease in T-cell receptor β clonotype diversity indicates that the immunotherapy not effective.

11. The method of claim 3, wherein the subject has cancer or has had cancer, and is receiving immunotherapy comprising check-point blockade agents.

12. The method of claim 11, further comprising determining that the subject is responding to immunotherapy if a statistically significant increase in frequency of one or more clonotypes is detected in the sample.

13. The method of claim 1, further comprising staining the CD4⁺ and tCD8⁺ cells isolated in step b) with FITC anti-human CD14 (clone M5E2), PE-Cy7 anti-human CD4 (clone SK3) and APC anti-human CD8 (clone RPA-T8; all BD Pharmingen) and sorting by FACS the CD4⁺ and tCD8⁺ cells isolated in step b).

14. The method of claim 1, wherein amplifying the cDNA further comprises using 5′ rapid amplification of cDNA ends (RACE) PCR.

15. The method of claim 1, wherein sequencing the cDNA further comprises deep sequencing the cDNA using Illumina miSEQ or Roche/454 platform.

16. The method of claim 1, wherein amplifying the cDNA further comprises amplifying the cDNA using a single oligonucleotide pair.

17. The method of claim 1, wherein step f) further comprises discarding sequences that are longer than 125 bp, does not have uncalled bases, has a phred quality score average above 30, or has an exact match to the TCRβ-constant primer or a multiplex identifier.

18. The method of claim 1, wherein at least one of the clonotypes of step g) is specific for an epitope on Epstein-Barr virus, or on CMV.

19. The method of claim 18, wherein at least one of the clonotypes is specific for the HLA-A2-restricted BMLF1₂₈₀ epitope from EBV.

20. The method of claim 1, wherein:
the subject has a T-cell disorder selected from the group comprising immunodeficiency, autoimmune disease, an infectious disease, inflammatory diseases, cancer, a precancerous condition; and
the method further comprises
i) obtaining a blood sample from a healthy subject, then processing the sample from the healthy subject according to steps b-g to determine the clonotype diversity of the sample from the healthy subject, and
j) determining that the subject having the T-cell disorder is immunocompromised if the clonotype diversity of the sample from the subject having the T-cell disorder is significantly lower than the clonotype diversity of the sample from the healthy subject.

21. The method of claim 1, wherein:
the subject has an autoimmune disease, and
the method further comprises
i) obtaining a blood sample from a healthy subject and then processing the sample from the healthy subject according to steps b-g to determine the clonotype frequency of the healthy sample, and
j) determining if the sample from the subject having the autoimmune disease shows a statistically significant increase of clonotype frequency of one or more clonotypes compared to the sample from the healthy subject.

22. The method of claim 21, further comprising, if the subject has a statistically significant increase of clonotype frequency of one or more clonotypes compared to the sample from the healthy subject, then treating the subject with immunosuppressants even if the subject is in remission.

23. The method of claim 1, wherein
the subject has a particular autoimmune disease, and
the method further comprises
i) obtaining blood samples from a plurality of other subjects having the same particular autoimmune disease, and from a healthy subject, and then processing the samples from the other subjects and the healthy subject according to steps b-g to determine the clonotype frequency of each sample,
j) determining if all of the samples from the subjects having the particular autoimmune disease show a statistically significant increase of clonotype frequency of one or more of the same clonotypes compared to the sample from the healthy subject, and
k) if the statistically significant increase of clonotype frequency of one or more of the same clonotypes compared to the sample from the healthy subject is detected, then determining that the one or more of the same clonotypes have expanded in the subjects having the particular autoimmune disease.

24. The method of claim 23, further comprising determining if there is a statistically significant correlation of the one or more of the same clonotypes with the particular autoimmune disease.

25. A method for identifying an agent that increases T-cell receptor β clonotype diversity, comprising
a) identifying a subject having a T-cell disorder selected from the group comprising immunodeficiencies, autoimmune diseases, infectious diseases, inflammatory diseases, cancer, and a precancerous condition,
b) obtaining a first blood sample from a healthy subject and a second blood sample from subject having the T-cell disorder,
c) isolating CD4⁺ and CD8⁺ cells or subsets thereof from the two samples,
d) culturing the cells isolated in step c) in the first sample in a control culture and the cells isolated in step c) from the second sample in a test culture that is contacted with a test agent under conditions that permit the test agent to affect T-cell receptor β clonotype diversity,
e) extracting total RNA from homogenates of each of the control culture and the test culture,
f) generating cDNA from the total RNA from each of the control culture and the test culture,
g) amplifying the cDNA from each of the control culture and the test culture,
h) sequencing the amplified cDNA from each of the control culture and the test culture,
i) selecting cDNA sequences that encode a clonotype of T-cell receptor β,
j) identifying T-cell receptor β clonotypes in the cDNA sequences and quantifying the diversity of the clonotypes and the clonotype frequency of each clonotype from each of the control culture and the test culture, and
k) determining if there is a statistically significant increase in T-cell receptor β clonotype diversity or frequency in the test culture compared to the control culture, and if there is a statistically significant increase, then selecting the test agent as one that increases T-cell receptor β clonotype diversity or frequency.
